(19) United States
(12) Patent Application Publication
(10) Pub. No.: US 2024/0100229 A1
(43) Pub. Date: Mar. 28, 2024

(54) SYSTEM AND METHOD FOR ISOLATION AND AUTOLOGOUS USE OF ALPHA 2M MOLECULES TO TREAT RESPIRATORY CONDITIONS

(71) Applicant: Astaria Global, LLC, Houston, TX (US)

(72) Inventors: Daniel J. Dreyfuss, Hampstead, MD (US); Stuart Shoemaker, Eagle, ID (US); William J. Cramer, Houston, TX (US); Michael D. Duvall, Houston, TX (US)

(21) Appl. No.: 18/535,841

(22) Filed: Dec. 11, 2023

Related U.S. Application Data
(63) Continuation-in-part of application No. 18/215,498, filed on Jun. 28, 2023.
Application No. 18/215,498 is a continuation of application No. 17/837,090, filed on Jun. 10, 2022, now Pat. No. 11,833,279.
(60) Provisional application No. 63/209,685, filed on Jun. 11, 2021.
(60) Provisional application No. 63/467,497, filed on May 18, 2023.
(60) Provisional application No. 63/541,116, filed on Sep. 28, 2023.

(51) Int. Cl. A61M 1/02; A61J 1/20; A61K 35/16; A61K 38/17; A61K 38/57; B01L 3/00

(52) U.S. Cl. CPC A61M 1/029; A61J 1/201; A61J 1/2062; A61J 1/2082; A61J 1/2096; A61K 35/16; A61K 38/1722; A61K 38/57; B01L 3/5021; B01L 3/50825; A61M 2202/0417; B01L 2300/0832; B01L 2400/0409; B01L 2400/0478

(57) ABSTRACT

A method for treating a respiratory condition of a patient with Alpha-2 Macroglobulin (α2M) molecules includes drawing whole blood, separating plasma containing α2M molecules from other components of the whole blood, isolating the α2M molecules from the other components of the plasma, and administering at least some of the isolated α2M molecules to the patient via inhalation.

### RELATED APPLICATIONS

This application is a continuation-in-part of U.S. patent application Ser. No. 18/215,498 entitled “SYSTEM AND METHOD FOR ISOLATING ALPHA 2M MOLECULES” filed Jun. 28, 2023 by David J. Lutz et al. (Attorney Docket No. JL1235.017); which is a continuation of U.S. patent application Ser. No. 17/837,090 entitled “SYSTEM AND METHOD FOR ISOLATING ALPHA 2M MOLECULES” filed Jun. 10, 2022 by David J. Lutz et al. (Attorney Docket No. JL1235.005); which claims the benefit of the priority date of U.S. Provisional Application 63/209,685 entitled “SYSTEM AND METHOD FOR ISOLATING a2M MOLECULES” filed Jun. 11, 2021 by David J. Lutz et al. (Attorney Docket No. JL1234.004); the disclosures of each of which are incorporated herein by reference for all purposes.

This application also claims the benefit of the priority date of each of U.S. Provisional Application 63/467,497 entitled “SYSTEM AND METHOD FOR ISOLATING AND USE OF ALPHA 2M MOLECULES IN TREATING RESPIRATORY CONDITIONS” filed May 18, 2023 by Daniel J. Dreyfuss et al. (Attorney Docket No. JL1235.013); and of U.S. Provisional Application 63/541,116 entitled “SYSTEM AND METHOD FOR ISOLATION AND ALLOGENEIC USE OF α2M MOLECULES TO TREAT MEDICAL CONDITIONS” filed Sep. 28, 2023 by Daniel J. Dreyfuss et al. (Attorney Docket No. JL1235.014); the disclosures of each of which are also incorporated herein by reference for all purposes.

### BACKGROUND

### 1. Technical Field

The present disclosure relates to medical procedures for the isolation, concentration and delivery of Alpha-2 Macroglobulin (α2M) molecules to treat respiratory conditions.

### 2. Description of the Related Art

For reasons that have remained unclear, rates of occurrence of various respiratory conditions, including asthma and obstructive pulmonary disease, have been observed to be increasing in recent years for a wide variety of creatures, including in various pets and farm animals (e.g., camels, dogs, horses, pigs, etc.). By way of example, in horses, rates of equine asthma (EA), including recurrent airway obstruction (RAO) and summer pasture-associated obstructive pulmonary disease (SPAOPD), have been observed to be increasing over the last couple of decades. These increasing rates also seem to mirror similar increasing rates of respiratory conditions in humans over a similar period of time.

Where humans are concerned, the increasing rates of respiratory conditions have already prompted increased efforts to discern the causes, and to develop treatments and/or cures. This has already resulted in an increasing variety of treatments available both over the counter and by prescription. A number of these treatments involve the delivery of a saline solution, and/or a solution of a pharmaceutical, in aerosolized form (e.g., through the use of a nebulizer). However, and regardless of the exact choice of delivery mechanism, many of such treatments are based on the use steroids that can have a variety of undesirable side effects.

Where horses are concerned, these increasing rates of occurrence of respiratory conditions such as EA have prompted a consensus in the veterinary community that there is now a pressing need for a similar increase in efforts to discern the causes, and to develop treatments and/or cures. Given the numerous biological similarities between humans and horses, and given the apparent similarities in increases in rates of respiratory conditions over a similar time period for both, a tendency has developed for treatments that were originally created for respiratory conditions in humans to be applied (at least experimentally) to treating respiratory conditions in horses. Unfortunately, this has also created at least the risk of having a similar variety of undesirable side effects in horses, such as those presented by the use of steroids.

Fortunately, medical study and research continues to reveal ever more detail about processes that occur in the bodies of human beings and other creatures, including details about the healing process that is triggered in response to injuries to a variety of tissues. By way of example, it has been found that injuries to at least respiratory tissues, whether caused by physical trauma, allergy response or disease, triggers a complex combination of both regenerative and destructive activities.

More specifically, there is a combination of growth of new tissue to repair and/or replace damaged tissue, and a remodeling of both old and new tissues to recreate a structure that was damaged with a correct shape and size. The remodeling process is effected by the provision of proteases at the location of the tissue damage to effectively “sculpt” the tissue structures that result from the growth process through the selective breakdown of the proteins making up portions of both old and new tissues. In effect, the healing process is meant to be a balance of both a growth process, and a selective destruction process in which excess portions of tissue are “trimmed” away.

Unfortunately, it is not uncommon for such “sculpting” to go too far as a result of an overabundance and/or hyperactivity of the proteases. The result may be misshapen tissue structures, the excessive formation of fibrotic tissue (e.g., scar tissue) in place of normal tissue, and/or the destruction of existing tissue that is not accompanied by replacement thereof.

In particular regarding respiratory conditions, the result may be misshapen air sac structures (e.g., enlarged and floppy air sac structures that are less effective), the excessive formation of fibrotic tissue (e.g., thickened air sac tissue), and/or the destruction of air sac tissue without repair (e.g., a reduction in the overall quantity of air sacs). By way of example, it may be that the inhalation of airborne allergens (e.g., fungi in a dusty feeding trough for horses), and/or particles having a microscopically sharp geometry that causes irritation (e.g., asbestos particles) triggers the inflammation of lung tissue, leading to a misperformed healing process in which the proteases become overactive. This may lead to thickening of air sac tissue that reduces the efficiency of blood oxygenation, and/or that causes the lungs to become less elastic such that physical act of breathing in and out requires more physical exertion.

An approach is needed to control the healing process for respiratory tissues to cause the healing process to proceed, as it should, with a better balance between the growth of new respiratory tissues and the “sculpting” of both old and new respiratory tissues. Also, at least in the case of larger animals, a need exists for such an approach to be performable with simpler equipment during house calls at locations distant from medical facilities.

### BRIEF SUMMARY

Technologies are described for more efficiently isolating α2M molecules in a non-laboratory setting for use in treating musculoskeletal conditions.

A method for treating a respiratory condition of a patient with Alpha-2 Macroglobulin (α2M) molecules includes: drawing whole blood, separating plasma containing α2M molecules from other components of the whole blood, isolating the α2M molecules from the other components of the plasma, and administering at least some of the isolated α2M molecules to the patient via inhalation.

A kit for treating a respiratory condition of a patient with Alpha-2 Macroglobulin (α2M) molecules includes at least one separator tube, wherein each separator tube of the at least one separator tube includes: an elongate transparent tube that defines an opening at one end that is sealed with a cap that is penetrable to receive whole blood; and an amount of separator gel disposed within the separator tube to cooperate with a first centrifugal force exerted on the separator tube for a first period of time during a first centrifuging stage to separate plasma containing α2M molecules from other components of the whole blood. The kit also includes at least one isolator, wherein each isolator of the at least one isolator includes: a filter; a first cylinder defined by a first cylindrical wall having a first end that is configured to be closable with a septum cap that is penetrable to receive the plasma containing the α2M molecules following the first centrifuging stage, and having a second end that is closed with the filter; and a second cylinder defined by a second cylindrical wall having a first end that is closed where the second cylindrical wall narrows to form a conically-shaped end portion, and having a second end that defines an opening that is configured to be coupled to the filter in a manner that causes a first interior space of the first cylinder and a second interior space of the second cylinder to be separated by the filter, wherein the filter is configured to cooperate with a second centrifugal force exerted on the isolator for a second period of time during a second centrifuging stage to isolate the α2M molecules from other components of the plasma. The further includes a transfer device that includes: a separator tube port configured to receive each separator tube of the at least one separator tube, one at a time, wherein the separator tube port comprises at least one hollow needle configured to penetrate the cap of each separator tube to couple the separator tube to a syringe port of the transfer device; and a syringe port configured to receive an end connector of a transfer syringe that is configured to be coupled to a transfer needle, wherein, following the first centrifuging stage and prior to the second centrifuging stage, while each separator tube of the at least one separator tube is coupled to the separator tube port, a plunger of the transfer syringe is operable to withdraw at least some of the plasma from within the separator tube and into the transfer syringe through the transfer device, and following transfer of plasma from each separator tube of the at least one separator tube, and with the transfer needle coupled to the end connector to penetrate the septum cap of each isolator of the at least one isolator, the plunger of the transfer syringe is operable to inject the plasma within the transfer syringe into the at least one isolator. The kit still further includes a nebulizer configured to be provided with the α2M molecules isolated during the second centrifuging stage, and to administer the α2M molecules to the patient via inhalation.

### BRIEF DESCRIPTION OF THE DRAWINGS

The disclosure will be better understood and when consideration is given to the drawings and the detailed description which follows. Such description makes reference to the annexed drawings wherein:

FIGS. 1A, 1B, 1C and 1D, together, provide an overview of aspects of differing embodiments of a system and method of isolating, concentrating and administering α2M molecules from whole blood in an autologous manner via inhalation.

FIGS. 2A, 2B, 2C, 2D, 2E, 2F, 2G, 2H, 2I, 2J and 2K, together, provide a more detailed presentation of aspects of an example of the enrichment system of FIG. 1B.

FIGS. 3A, 3B, 3C and 3D, together, provide a more details presentation of aspects of an example of the inhalation system of any of FIGS. 1A-D.

FIGS. 4A, 4B and 4C, together, provide a flow chart of an example of the system and method of FIG. 1B.

FIGS. 5A, 5B and 5C, together, provide a flow chart of an example of the system and method of FIG. 1D.

### DETAILED DESCRIPTION

In the following detailed description, reference is made to the accompanying drawings that form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Broadly speaking, disclosed herein is a system and a method for isolating and concentrating α2M molecules from whole blood, and then administering those α2M molecules to a patient via a nebulizer to treat a respiratory condition. More specifically, the isolating and concentration of a2M molecules may entail the use of a centrifuge together with a set of separator tubes and one or more isolators to perform two stages of centrifugation. The administration of the isolated and concentrated α2M molecules may entail the preparation and use of a solution of those α2M molecules with the nebulizer in an autologous manner. The patient may be any of a wide variety of creature, including and not limited to, a camel, a dog, a horse, a human, a pig, etc.

A method for treating a respiratory condition of a patient with Alpha-2 Macroglobulin (α2M) molecules includes: drawing whole blood, separating plasma containing α2M molecules from other components of the whole blood, isolating the α2M molecules from the other components of the plasma, and administering at least some of the isolated α2M molecules to the patient via inhalation.

A kit for treating a respiratory condition of a patient with Alpha-2 Macroglobulin (α2M) molecules includes at least one separator tube, wherein each separator tube of the at least one separator tube includes: an elongate transparent tube that defines an opening at one end that is sealed with a cap that is penetrable to receive whole blood; and an amount of separator gel disposed within the separator tube to cooperate with a first centrifugal force exerted on the separator tube for a first period of time during a first centrifuging stage to separate plasma containing α2M molecules from other components of the whole blood. The kit also includes at least one isolator, wherein each isolator of the at least one isolator includes: a filter; a first cylinder defined by a first cylindrical wall having a first end that is configured to be closable with a septum cap that is penetrable to receive the plasma containing the α2M molecules following the first centrifuging stage, and having a second end that is closed with the filter; and a second cylinder defined by a second cylindrical wall having a first end that is closed where the second cylindrical wall narrows to form a conically-shaped end portion, and having a second end that defines an opening that is configured to be coupled to the filter in a manner that causes a first interior space of the first cylinder and a second interior space of the second cylinder to be separated by the filter, wherein the filter is configured to cooperate with a second centrifugal force exerted on the isolator for a second period of time during a second centrifuging stage to isolate the α2M molecules from other components of the plasma. The further includes a transfer device that includes: a separator tube port configured to receive each separator tube of the at least one separator tube, one at a time, wherein the separator tube port comprises at least one hollow needle configured to penetrate the cap of each separator tube to couple the separator tube to a syringe port of the transfer device; and a syringe port configured to receive an end connector of a transfer syringe that is configured to be coupled to a transfer needle, wherein, following the first centrifuging stage and prior to the second centrifuging stage, while each separator tube of the at least one separator tube is coupled to the separator tube port, a plunger of the transfer syringe is operable to withdraw at least some of the plasma from within the separator tube and into the transfer syringe through the transfer device, and following transfer of plasma from each separator tube of the at least one separator tube, and with the transfer needle coupled to the end connector to penetrate the septum cap of each isolator of the at least one isolator, the plunger of the transfer syringe is operable to inject the plasma within the transfer syringe into the at least one isolator. The kit still further includes a nebulizer configured to be provided with the α2M molecules isolated during the second centrifuging stage, and to administer the α2M molecules to the patient via inhalation.

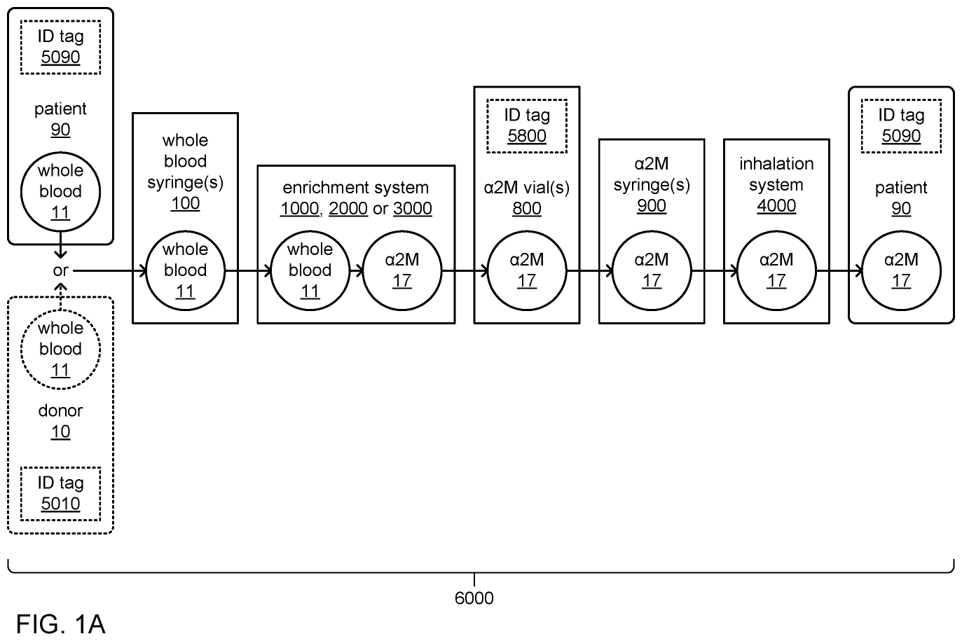

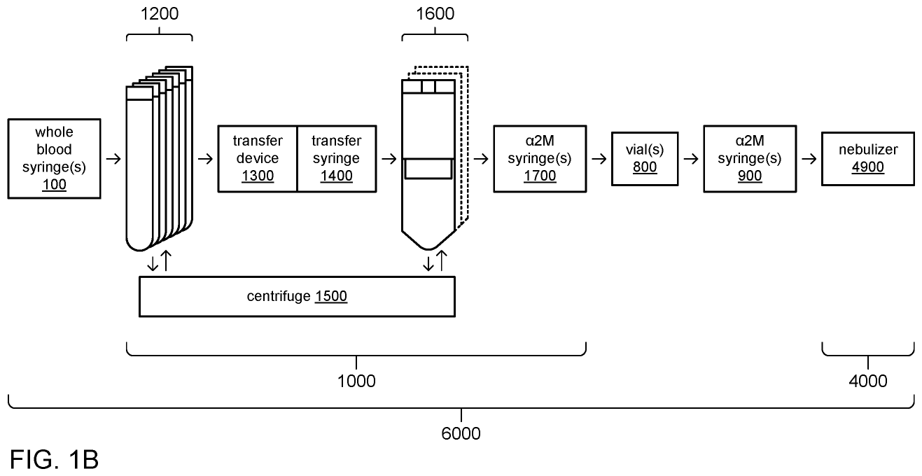

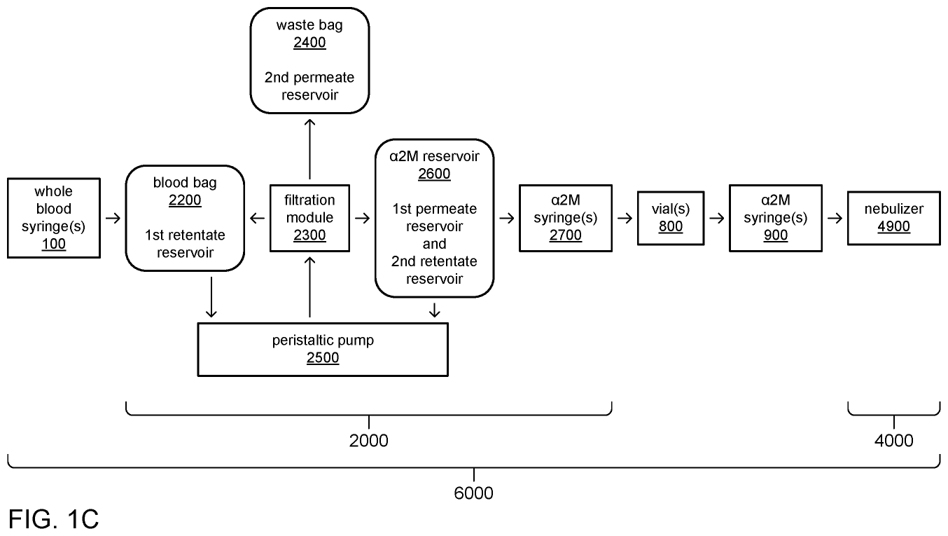

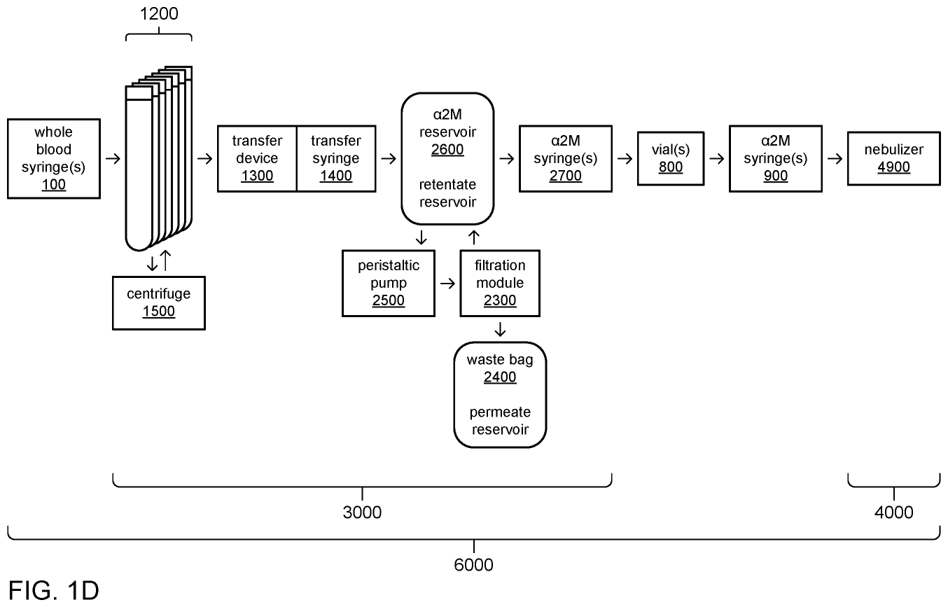

Turning to FIG. 1A, a system 6000 for the isolation, concentration and administration of α2M molecules 17 includes an enrichment system 1000, 2000 or 3000 to perform the isolation and concentration functions from whole blood 11. As will be described in greater detail, the enrichment system 1000 may employ a combination of a centrifuge, separator gel and filter to isolate the α2M molecules 17 from the whole blood 11. However, as will also be described in greater detail, the enrichment system 2000 may employ a combination of a peristaltic pump and multiple filters to isolate the α2M molecules 17 from the whole blood 11; and the enrichment system 3000 may employ a hybrid combination of components of the enrichment systems 1000 and 2000 to do so. The system 6000 also includes an inhalation system 4000 by which the α2M molecules 17 may be administered in an autologous manner to a patient 90 via inhalation. The system 6000 may further include a tracking system 5000 to control the administration function.

Within each enrichment system 1000, 2000 or 3000, various techniques are employed to separate α2M molecules 17 from other components of that whole blood 11, to concentrate the α2M molecules 17, and to provide the concentrated α2M molecules 17 as multiple aliquots in separate ones of a set of multiple vials 800. From each such vial 800 in such a set, one or more α2M syringes 900 and a nebulizer of the inhalation system 4000 may be used in administering the α2M molecules 17 derived from the whole blood 11 to the patient 90. As also depicted, it may be that the patient 90 and the α2M vials 800 are provided with identifier tags 5090 and 5800, respectively, of the inhalation system 4000 to enable tracking of at least patients 90 and the α2M vials 800 from which the α2M molecules 17 may be administered thereto.

Thus, as depicted, one or more whole blood syringes 100 may be used to draw whole blood 11 from the patient 90, and to provide the whole blood 11 as an input to one of the enrichment systems 1000, 2000 or 3000. And thus, it is envisioned that the α2M molecules 17 that are derived from the whole blood 11 are used in an autologous manner. However, and as also depicted, other embodiments are possible in which the whole blood 11 may be drawn from a donor 10 who may be the patient 90, but may be of the same species as the patient 90. Thus, in such other embodiments, it may be that the α2M molecules 17 that are derived from such whole blood 11 are used allogeneically. As still another embodiment, it may be that the whole blood 11 is drawn from a donor 10 who is not the patient 90, and who is also not of the same species as the patient 90. Thus, in such still other embodiments, it may be that the α2M molecules 17 that are derived from such whole blood 11 are used xenogeneically. As further depicted, where the whole blood 11 is drawn from a donor 10 who is an individual other than the patient 90 (whether of the same species as the patient 90, or not), the donor 10 may be provided with a different identifier tag 5010 from the identifier tag 5090 of the patient 90.

It should be further noted that, while it is envisioned that the α2M molecules 17 are to be isolated from whole blood 11 drawn from the patient 90 to which the α2M molecules 17 are to be administered (or from a donor 10, as just discussed), in other embodiments, it may be that at least some of the α2M molecules 17 are artificially generated. By way of example, it may be that specialized cells, bacteria or still another form of microbe generates such α2M molecules 17 as part of a process performed in a lab or other facility. Such α2M molecules 17 and/or the microbes that generate them may be at least partially created through use of an artificially generated genetic sequence, and/or may be created through a selective breeding process. In some of such embodiments, at least a portion of the genetic sequence used may be in some manner derived from a genetic sequence of the patient 90.

By way of example, a virus or other mechanism may be used to introduce at least a portion of a genetic sequence of the patient 90 into cells, bacteria or still another form of microbe to cause generation of the α2M molecules 17 with one or more characteristics (e.g., blood type compatibility) that in some way enhances acceptance by the patient 90 and/or reduces an immune response of the patient 90. Such an approach may be used where a patient 90 has a relatively small amount and/or density of α2M molecules 17 within their whole blood 11 such that isolation and concentration of their own α2M molecules 17 for autologous use is at least impractical, if not impossible. It may be that such artificially generated α2M molecules 17 may be used to augment α2M molecules 17 that are generated from the whole blood 11 of the patient 90 in support of such autologous use.

FIG. 1B, together with FIG. 1A, provide a high level overview of the enrichment system 1000, and the process of using it. The isolation of α2M molecules 17 begins with the use of the one or more whole blood syringes 100 to draw an amount of whole blood 11 from the patient 90 (or donor 10), and then to transfer portions of that whole blood 11 into each of multiple separator tubes 1200. The set of separator tubes 1200 may then be placed within the centrifuge 1500 to be subjected to centrifugal force for a first predetermined period of time (i.e., a first stage of centrifuging) to cause separation of the blood plasma containing the α2M molecules 17 from other components of the whole blood 11 with the aid of a separator gel incorporated into each of the separator tubes 1200. Following such separation of the plasma from the other blood components, one or both of a transfer device 1300 and a transfer syringe 1400 may then be used to retrieve the separated blood plasma from each of the separator tubes 1200, and transfer that plasma to one or more isolators 1600. The one or more isolators 1600 may then be placed within the centrifuge 1500 to be subjected to centrifugal force for a second predetermined period of time (i.e., a second stage of centrifuging) to cause isolation and concentration of the α2M molecules 17 from the plasma with the aid of a membrane filter incorporated into each isolator 1600. Following such isolation and concentration of the α2M molecules 17, one or more α2M syringes 1700 may then be used to retrieve the α2M molecules 17 from the one or more isolators 1600, and transfer the α2M molecules 17 to a set of multiple ones of the vials 800, thereby creating a set of aliquots of the α2M molecules 17. Following such division of the α2M molecules 17 into aliquots, one or more of the vials 800 may then be frozen, before being distributed for being subsequently administered to the patient 90 via inhalation using a combination of the α2M syringe(s) 900 and the nebulizer 4900 of the inhalation system 4000.

As is about to become apparent, the enrichment system 1000, unlike the enrichment systems 2000 and 3000 that are about to be described, entails the use of relatively small, lightweight and inexpensive components and devices. In particular, unlike the enrichment systems 2000 and 3000 that are about to be described, the enrichment system 1000 does not include a peristaltic pump. Thus, as will be appreciated by those skilled in the art, it is significantly more feasible to bring the components and devices of the enrichment system 1000 on a house call to a farm or other location at which larger animals may be kept, and away from a medical facility. Additionally, the enrichment system 1000 enables the isolation and concentration of α2M molecules 17 from the whole blood 11 in less time than is possible using either of the enrichment systems 2000 or 3000.

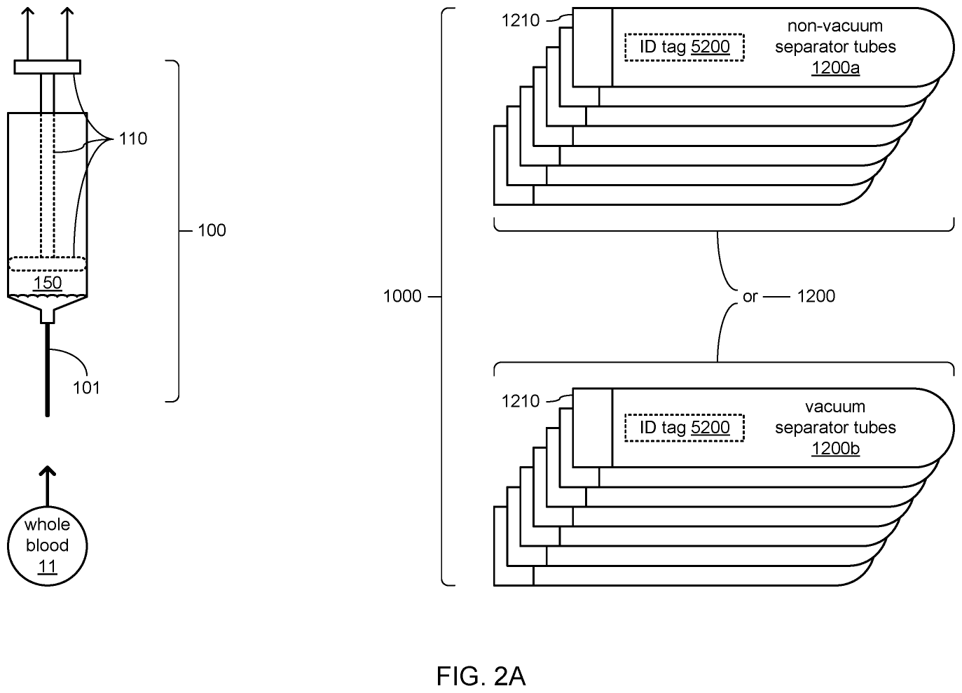

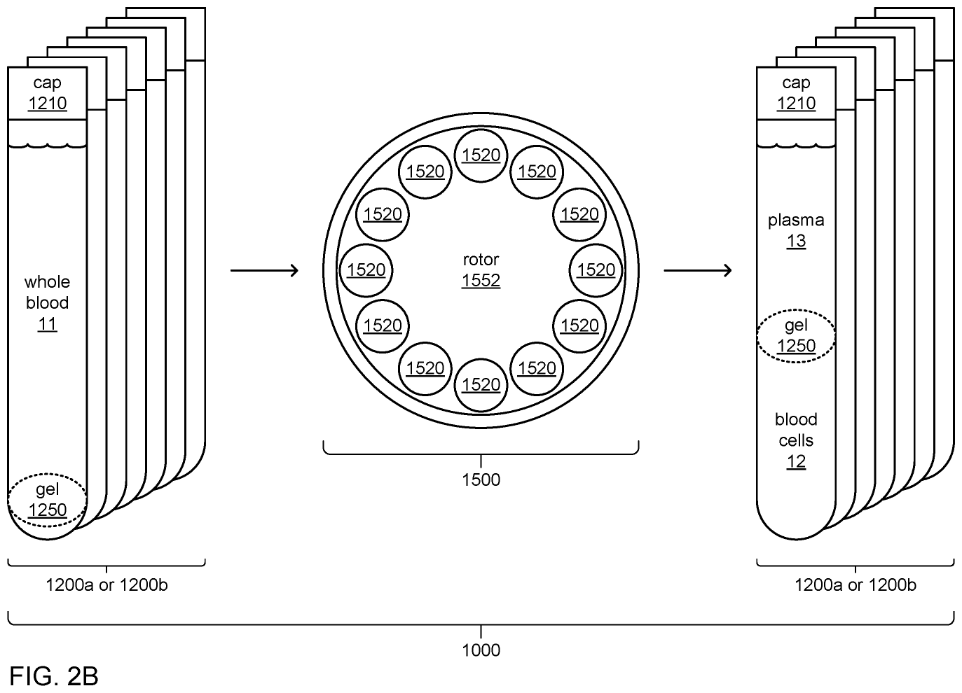

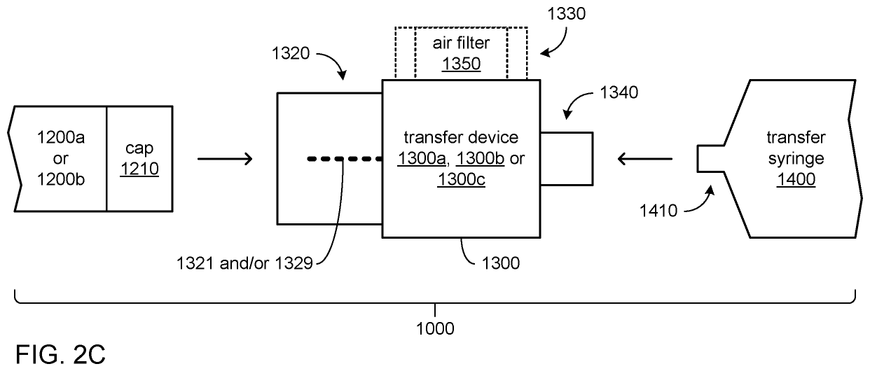

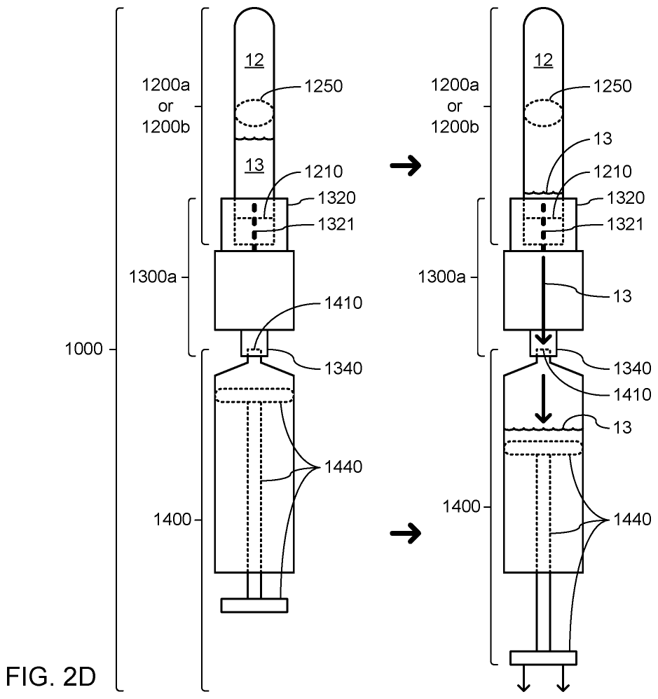

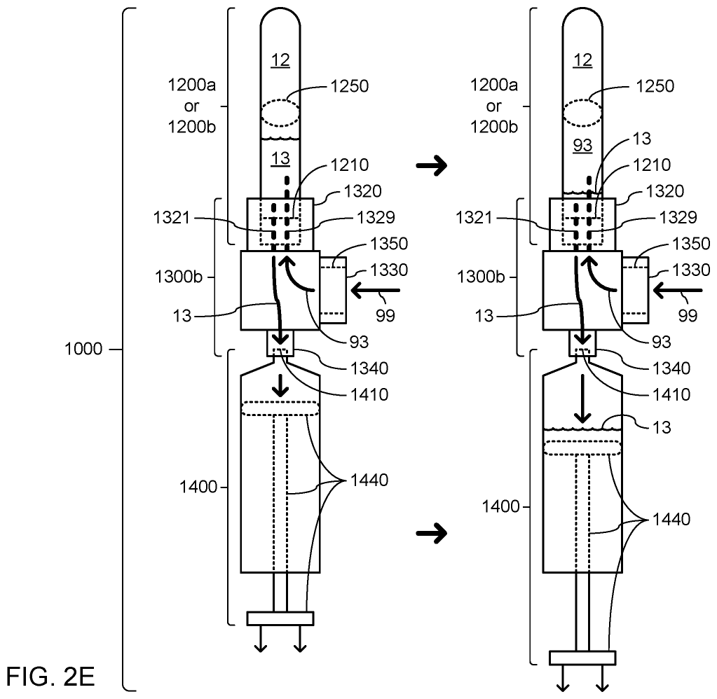

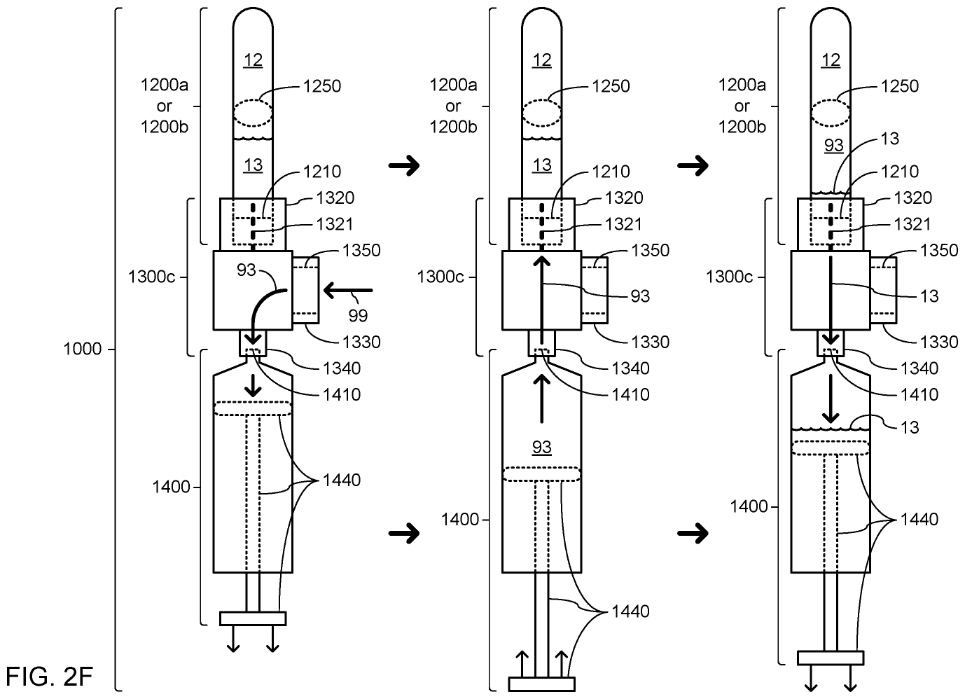

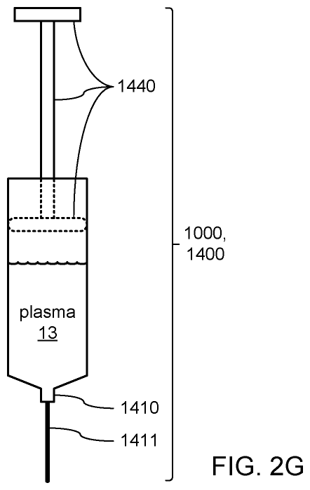

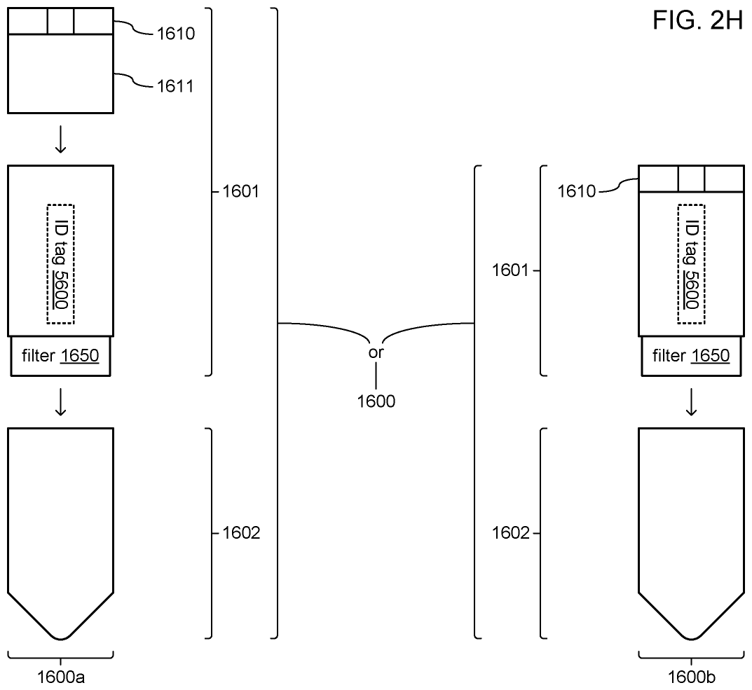

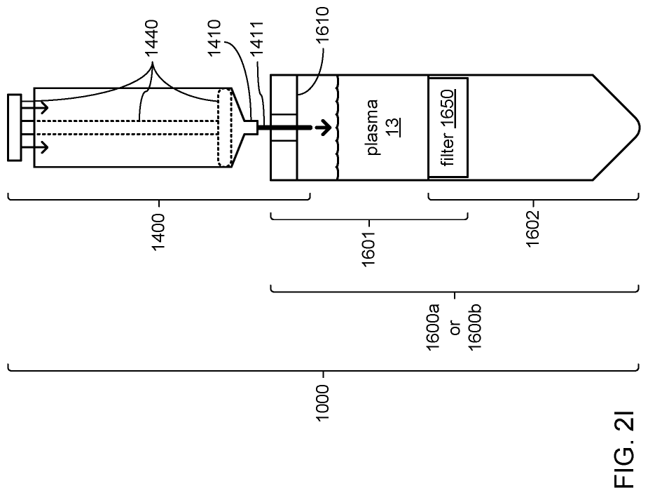

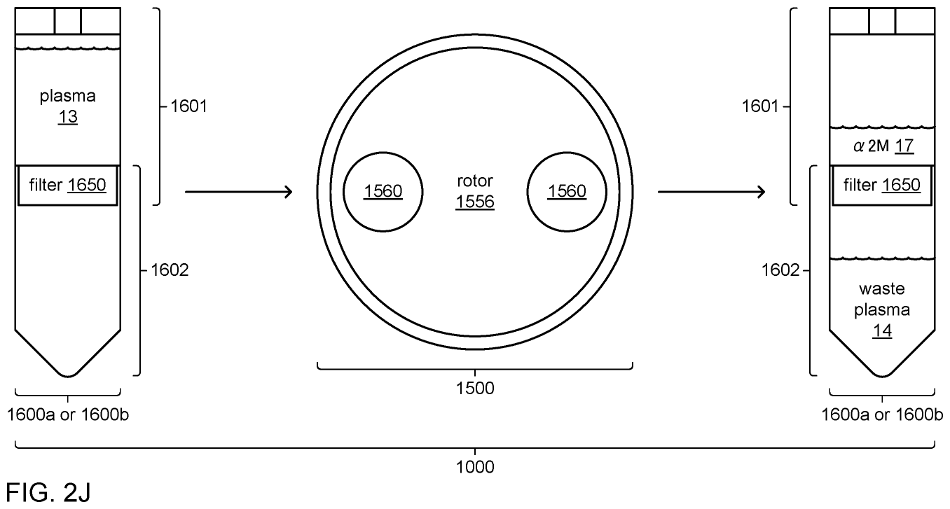

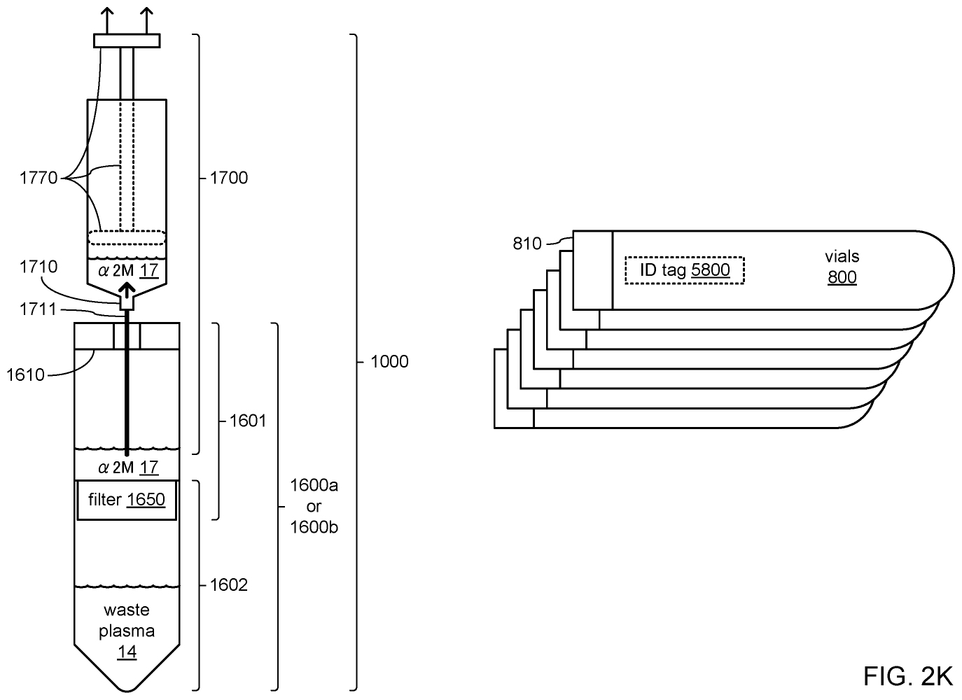

The enrichment system 1000 is depicted and described in greater detail in FIGS. 2A-J.

FIG. 1C, together with FIG. 1A, provide a high level overview of the enrichment system 2000, and the process of using it. The isolation of α2M molecules 17 begins with the use of the one or more whole blood syringes 100 to draw an amount of whole blood 11 from the patient 90 (or donor 10), and then to transfer that whole blood 11 into a blood bag 2200. A set of tubes that interconnect the blood bag 2200, a filtration module 2300, a waste bag 2400, and an α2M reservoir 2600 may then be fitted to a peristaltic pump 2500. Operation of the peristaltic pump 2500 may squeeze at least one of such tubes in a manner causing flow(s) of whole blood 11 and blood components among the blood bag 2200, the filtration module 2300, the waste bag 2400 and the α2M reservoir 2600 that brings about the isolation and concentration of the α2M molecules 17 within the α2M reservoir 2600. Following such isolation and concentration of the α2M molecules 17, one or more α2M syringes 2700 may then be used to retrieve the α2M molecules 17 from the α2M reservoir 2600, and transfer the α2M molecules 17 to the set of vials 800, thereby creating a set of aliquots of the α2M molecules 17. Following such division of the α2M molecules 17 into aliquots, one or more of the vials 800 may then be frozen, before being distributed for being subsequently administered to the patient 90 via inhalation using a combination of the α2M syringe(s) 900 and the nebulizer 4900 of the inhalation system 4000.

FIG. 1D, together with FIG. 1A, provide a high level overview of the enrichment system 3000, and the process of using it. As previously mentioned, the enrichment system 3000 is a hybrid of portions of each of the enrichment systems 1000 and 2000. More specifically, in the enrichment system 3000, the separation of plasma including the separation of the blood plasma containing the α2M molecules 17 from other components of the whole blood 11 is performed in a manner similar to how blood plasma containing the α2M molecules 17 is separated from other components of the whole blood 11 in the enrichment system 1000. However, the isolation of the α2M molecules 17 from other components of the blood plasma, and the concentration of the α2M molecules 17, are both performed in a manner similar to how α2M molecules 17 are so isolated and concentrated in the enrichment system 2000.

Thus, in a manner similar to the enrichment system 1000 of FIG. 1B, the isolation of α2M molecules 17 begins with the use of the one or more whole blood syringes 100 to draw an amount of whole blood 11 from the patient 90 (or donor 10), and then to transfer portions of that whole blood 11 into each of multiple separator tubes 1200. Again, the set of separator tubes 1200 may then be placed within the centrifuge 1500 to be subjected to centrifugal force for a predetermined period of time to cause separation of the blood plasma containing the α2M molecules 17 from other components of the whole blood 11 with the aid of the separator gel incorporated into each of the separator tubes 1200.

Following the separation of the plasma from the other blood components, and in a manner similar to the enrichment system 2000 of FIG. 1C, one or both of a transfer device 1300 and a transfer syringe 1400 may then be used to retrieve the separated blood plasma from each of the separator tubes 1200, and transfer that plasma to an α2M reservoir 2600. One of such a set of tubes that interconnect the filtration module 2300, a waste bag 2400, and the α2M reservoir 2600 may then be fitted to the peristaltic pump 2500. Operation of the peristaltic pump 2500 may squeeze such a tube in a manner causing a flow of plasma and components of the plasma among the filtration module 2300, the waste bag 2400 and the α2M reservoir 2600 that brings about the isolation and concentration of the α2M molecules 17 within the α2M reservoir 2600. Following such isolation and concentration of the α2M molecules 17, one or more α2M syringes 2700 may then be used to retrieve the α2M molecules 17 from the α2M reservoir 2600, and transfer the α2M molecules 17 to the set of vials 800, thereby creating a set of aliquots of the α2M molecules 17. Following such division of the α2M molecules 17 into aliquots, one or more of the vials 800 may then be frozen, before being distributed for being subsequently administered to the patient 90 via inhalation using a combination of the α2M syringe(s) 900 and the nebulizer 4900 of the inhalation system 4000.

FIGS. 2A-K, taken together, present aspects of the enrichment system 1000 in greater detail. More specifically, FIGS. 2A-B depict aspects of the separation of plasma 13 containing the α2M molecules 17 from other components of the whole blood 11; FIGS. 2C-I depict aspects of transferring the plasma 13 from the separator tubes 1200 to the isolator(s) 1600; and FIGS. 2J-2K depict aspects of isolating and concentrating the α2M molecules 17.

Turning to FIG. 2A, as depicted, in the enrichment system 1000, the set of separator tubes 1200 may be either a set of non-vacuum separator tubes 1200a or a set of vacuum separator tubes 1200b. Each of the different types of separator tube 1200a and 1200b may be an elongate transparent tube with a single opening on one end that is sealed with a cap 1210 to at least maintain sterile conditions therein. The cap 1210 may be formed from a relatively flexible material that enables a hollow needle to penetrate therethrough for transferring gases and/or fluids into and/or out of the interior of each of the separator tubes 1200a or 1200b in a manner in which a seal is maintained around such a needle. Such a flexible material may also be self-sealing in a manner that causes a re-sealing of holes formed therethrough by the penetration and subsequent removal of such a needle.

In embodiments of the enrichment system 1000 that include the set of vacuum separator tubes 1200b, each of the vacuum separator tubes 1200b may be a VACUTAINER® tube of a type offered by Becton, Dickson and Company of Franklin Lakes, New Jersey, USA. As will be familiar to those skilled in the art, each such vacuum separator tube 1200b, in its new and unused condition, may be pre-provided with a vacuum therein that the seal provided by the cap 1210 is used to maintain.

Regardless of which of the separator tubes 1200a or 1200b are used, the quantity of separator tubes 1200a or 1200b that are used may vary based on such factors as the volume of whole blood 11 that may be safely drawn from the patient 90 (or donor 10), the type and/or severity of the respiratory condition that is to be treated, and/or the maximum quantity of separator tubes 1200a or 1200b that may be used with the centrifuge 1500 at a time. As those skilled in the art will readily recognize, the volume of whole blood 11 that may be safely drawn from the patient 90 (or donor 10) may depend on at least the species of the patient 90 (or donor 10), which again, may include and not be limited to, a camel, a dog, a horse, a human, a pig, etc. Thus, in embodiments of the system 6000 that incorporate the enrichment system 1000, it is contemplated that the system 6000 may be offered in differently-sized variants of kits, such as a smaller variant of kit that may include 1 to 4 separator tubes 1200a or 1200b, a mid-sized variant of kit that may include 5 to 8 separator tubes 1200a or 1200b, and/or a larger variant of kit that may include 9 to 16 (or still more) separator tubes 1200a or 1200b.

A plunger 110 of the whole blood syringe 100 may be operated to draw whole blood 11 from a blood vessel of the patient 90 (or donor 10) and into the whole blood syringe 100 via a needle 101 thereof. The whole blood syringe 100 may include a human-readable scale by which the volume of whole blood that is drawn is able to be measured as the plunger 110 is so operated to ensure that just the amount of whole blood 11 that is needed for the chosen quantity of separator tubes 1200a or 1200b is successfully drawn. After the appropriate volume of whole blood 11 is drawn, the whole blood syringe 100 may then be used to inject a portion of the drawn whole blood 11 into each of the separator tubes 1200a or 1200b through the cap 1210 via the needle 101.

As additionally depicted, in some embodiments, and prior to being used to draw whole blood 11 from the patient 90 (or donor 10), the whole blood syringe 100 may be partially pre-filled (e.g., by the nurse, medical technician, veterinarian technician, doctor, veterinarian, etc.) with an amount of an anticoagulent 150, such as a citrate dextrose solution (ACD-A), to prevent the drawn whole blood from coagulating therein.

As also additionally depicted, each of the separator tubes 1200a or 1200b may carry an identifier (ID) tag 5200 that is indicative of identity of the patient 90 (or donor 10) to associate the whole blood 11 therein with the individual from which it was drawn. In some embodiments, it may be that the ID tags 5200 are stickers that carry a one-dimensional or two-dimensional bar code that is associated with the patient 90 (or donor 10). It may be that such stickers are printed on or about the time that the whole blood 11 is drawn from the patient 90 (or donor 10) as part of a procedure that is meant to ensure that each of those particular ID tags 5200 does indeed carry a bar code indicative of the identity of the patient 90 (or donor 10), and that the set of separator tubes 1200a or 1200b to which those particular ID tags 5200 are applied are indeed caused to contain the whole blood 11 of the patient 90 (or donor 10). In other embodiments, it may be that the ID tags 5200 are radio frequency identification (RFID) tags that store data serving as an identifier of the patient 90 (or donor 10). It may be that such an identifier is caused to be stored within such RFID tags on or about the time that the whole blood 11 is drawn from the patient 90 (or donor 10) as part of a procedure that is meant to ensure that each of those particular RFID tags does indeed store an identifier associated with the patient 90 (or donor 10), and that the set of separator tubes 1200a or 1200b that carry those particular RFID tags is indeed caused to contain the whole blood 11 of the patient 90 (or donor 10).

Turning to FIG. 2B, each of the different types of separator tube 1200a and 1200b may include (at least in its new and unused condition) a small amount of a separator gel 1250 disposed toward the end opposite the end that is closed with the cap 1210. Thus, as depicted, following the collection and storage of the whole blood 11 among the set of separator tubes 1200a or 1200b, as described above in reference to FIG. 2A, the portion of the whole blood 11 within each of the separator tubes 1200a or 1200b may be disposed therein between the cap 1210 at one end and the separator gel 1250 at the other end.

With the set of separator tubes 1200a or 1200b so filled with portions of whole blood 11, the set of separator tubes 1200a or 1200b may be placed within the centrifuge 1500 to be subjected to centrifugal force for a first period of time that is deemed sufficient to fully separate the plasma 13 thereof from the red and white blood cells 12 thereof. More specifically, and as depicted, the centrifuge 1500 may be used in conjunction with the separator gel 1250 to effect such a separation of components of the whole blood 11. Thus, when such isolation of the plasma 13 is complete, the separator gel 1250 within each of the separator tubes 1200a or 1200b should occupy a position that physically separates the plasma 13 from the red and white blood cells 12, thereby preventing these blood components 12 and 13 from becoming mixed together, again.

As depicted, and as will be familiar to those skilled in the art, the centrifuge 1500 may include a rotor 1552 that defines a set of holding positions 1520 that each have a shape and dimensions selected to hold a tube of matching shape and dimensions, such as one of the separator tubes 1200a or 1200b. As also depicted, it may be that the quantity and placement of such holding positions 1520, as defined by the rotor 1552, may be selected to enable various quantities of such tubes to be distributed among the holding positions 1520 in a manner that distributes the weight thereof in a balanced manner that enables relatively smooth operation of the centrifuge 1500.

As will also be familiar to those skilled in the art, it may be that the depicted rotor 1552 is exchangeable with one or more other rotors to thereby enable the centrifuge 1500 to be reconfigured to work with various different quantities and/or combinations of various tubes and/or other varieties of containers of differing shapes and/or sizes. Alternatively or additionally, it may be that the centrifuge 1500 is fitted with (or otherwise includes) a rotor with 2 or more “buckets.” Each such bucket may be able to be fitted with any of a variety of differing types of holder that may each be designed to provide holding position(s) for a differing quantity of and/or combination of various tubes and/or other varieties of containers of differing shapes and/or sizes.

Turning to FIG. 2C, as depicted, in some embodiments of the enrichment system 1000, the transfer device 1300 may be a single-flow device 1300a. Alternatively, in other embodiments of the enrichment system 1000, the transfer device 1300 may be a dual-flow device 1300b. In still other embodiments of the enrichment system 1000, the transfer device 1300 may be a three-way valve 1300c.

Each of the different types of transfer device 1300a, 1300b and 1300c may incorporate at least the depicted combination of a separator tube port 1320 and a syringe port 1340. Each of the different types of transfer device 1300b and 1300c may additionally incorporate a filtered air port 1330. As is about to be described, each of the different types of transfer device 1300a, 1300b and 1300c is configured to enable plasma 13 to be transferred from a separator tube 1200a or 1200b coupled to the separator tube port 1320, and to a transfer syringe 1400 coupled to the syringe port 1340. Additionally, and as is also about to be described, each of the different types of transfer device 1300b and 1300c is additionally configured to also enable external air surrounding the transfer device 1300b or 1300c to be drawn in through an air filter 1350 at the filtered air port 1330, and conveyed to the separator tube 1200a or 1200b that coupled to the separator tube port 1320.

It is envisioned that the interior volume of the transfer syringe 1400 is sufficiently large that all of the sum total of the amounts of the plasma 13 isolated within all of the separator tubes 1200a or 1200b (as a result of being subjected to centrifugal force by the centrifuge 1500, as earlier described) is able to be combined and retained within the transfer syringe 1400. As a result, it is envisioned that the transfer syringe 1400 is to remain connected to the syringe port 1340 by its end connector 1410 throughout the time that the plasma 13 is being transferred from each of the separator tubes 1200a or 1200b, and into the transfer syringe 1400.

FIG. 2D depicts aspects of the manner in which the single-flow device 1300a enables a transfer of plasma 13 out of the separator container 1200a or 1200b, and into the transfer syringe 1400. As depicted, the separator tube port 1320 may incorporate a plasma needle 1321 that is positioned to penetrate through the cap 1210 of a separator tube 1200a or 1200b to enable the flow through each of gases and/or liquids out of such a separator tube 1200a or 1200b. As also depicted, the syringe port 1340 may be configured to form a connection with an end connector 1410 carried at one end of the transfer syringe 1400.

As depicted, with a separator tube 1200a or 1200b coupled to the separator tube port 1320 such that the plasma needle 1321 penetrates the cap 1210 thereof, and with the end connector 1410 of the transfer syringe 1400 coupled to the syringe port 1340, there may be an initial equalization of pressures thereamong. However, it has been discovered by that the operation of the single-flow transfer device 1300a is not significantly changed by whether non-vacuum separator tubes 1200a or vacuum separator tubes 1200b are coupled to the separator tube port 1320. Thus, regardless of which type of separator tube 1200a or 1200b is coupled to the separator tube port 1320, pulling the plunger 1440 of the transfer syringe 1400 in a direction away from the end connector 1410 thereof may draw plasma 13 from within the separator tube 1200a or 1200b, and into the transfer syringe 1440, via the plasma needle 1321 and the end connector 1410.

FIG. 2E depicts aspects of the manner in which the dual-flow device 1300b enables a simultaneous transfer of filtered air 93 into a separator container 1200a or 1200b, and of plasma 13 out of the separator container 1200a or 1200b as part of transferring plasma 13 to the transfer syringe 1400. As depicted, the separator tube port 1320 may incorporate both an air needle 1329 and a plasma needle 1321 that are each positioned to penetrate through the cap 1210 of a separator tube 1200a or 1200b to enable the flow through each of gases and/or liquids into and/or out of such a separator tube 1200a or 1200b. Again, the syringe port 1340 may be configured to form a connection with an end connector 1410 carried at one end of the transfer syringe 1400.

As depicted, with a separator tube 1200a or 1200b coupled to the separator tube port 1320 such that the needles 1321 and 1329 penetrate the cap 1210 thereof, and with the end connector 1410 of the transfer syringe 1400 coupled to the syringe port 1340, there may be an initial equalization of pressures thereamong. More specifically, and especially where a vacuum separator tube 1200b is coupled to the separator tube port 1320, external air 99 may be drawn into the dual-flow device 1300a through the air filter 1350 of the filtered air port 1330, and then the resulting filtered air 93 may be conveyed into a separator tube 1200a or 1200b at the separator tube port 1320 via the air needle 1329. Pulling the plunger 1440 of the transfer syringe 1400 in a direction away from the end connector 1410 thereof may then draw plasma 13 from within the separator tube 1200a or 1200b, and into the transfer syringe 1440, via the plasma needle 1321 and the end connector 1410. In turn, more filtered air 93 may be drawn into the separator tube 1200a or 1200b to replace the plasma 13 that is so drawn out.

FIG. 2F depicts aspects of the manner in which the three-way valve 1300c enables a selective transfer of filtered air 93 into a separator container 1200a or 1200b, and of plasma 13 out of the separator container 1200a or 1200b as part of transferring plasma 13 to the transfer syringe 1400. As depicted, the separator tube port 1320 of the three-way valve 1300b may incorporate just a single plasma needle 1321 that is positioned to penetrate through the cap 1210 of a separator tube 1200a or 1200b to enable the flow therethrough of gases and/or liquids into and/or out of such a separator tube 1200a or 1200b. Again, the syringe port 1340 may be configured to form a connection with an end connector 1410 carried at one end of the transfer syringe 1400.

The three-way valve 1300c may incorporate a manually-operable valve (not specifically shown) of a type that is operable between at least two positions, where each position of the at least two positions causes one of the three ports 1320, 1330 or 1340 to be closed off from the other two of these two ports, while allowing gases and/or liquids to flow freely between the other two.

As depicted, for each separator tube 1200a or 1200b that is connected to the separator tube port 1320, the transfer of plasma 13 therefrom, and into the transfer syringe 1400, may begin with the three-way valve 1300b being operated to close off the separator tube port 1320, thereby connecting the syringe port 1340 to the filtered air port 1330. With the separator tube port 1320 so closed off, the plunger 1440 of the transfer syringe 1400 may be operated to draw filtered air 93 into the transfer syringe 1400. More precisely, the plunger 1440 of the transfer syringe 1400 may be operated to cause external air 99 that surrounds the three-way valve 1300b to be drawn in through the air filter 1350, thereby being filtered to become the filtered air 93 that is drawn into the transfer syringe 1400.

With an amount of such filtered air 93 now within the transfer syringe 1400, the three-way valve 1300b may then operated to close off the filtered air port 1330, thereby connecting the syringe port 1340 to the separator tube port 1320. With the filtered air port 1330 so closed off, the plunger 1440 of the transfer syringe 1400 may be operated to send filtered air 93 out of the transfer syringe 1400, through the three-way valve 1300b, through the plasma needle 1321, and into the separator tube 1200a or 1200b that is coupled to the separator tube port 1320. With filtered air 993 so conveyed into the separator tube 1200a or 1200b, the plunger 1440 of the transfer syringe 1400 may then be operated to draw most, if not all, of the plasma 13 out of the separator tube 1200a or 1200b, through the plasma needle 1321, through the three-way valve 1300b, and into transfer syringe 1400.

Referring back to each of FIGS. 2D-F, it should be noted, that such transfers of plasma 13 from the separator tubes 1200a or 1200b, and into the transfer syringe 1400 may need to be performed with the depicted combination of the separator tube 1200a or 1200b, the transfer device 1300a/1300b/1300c, and the transfer syringe 1400 held in an orientation in which the separator tube 1200a or 1200b is at a higher elevation than the transfer syringe 1400.

Turning FIG. 2G, following the transfer of plasma 13 out of each of the separator tubes 1200a or 1200b, and into the transfer syringe 1400, the transfer syringe 1400 may then be disconnected from the transfer device 1300a, 1300b or 1300c. Then, a transfer needle 1411 may be connected to the end connector 1410 of the transfer syringe 1400 in preparation for injecting the plasma 13 into the isolator 1600.

Turning to FIG. 2H, as depicted, the isolator 1600 may include a combination of a first cylinder 1601 and a second cylinder 1602. Both of these cylinders 1601 and 1602 may be of a generally elongate shape defining a pair of ends.

One end of the first cylinder 1601 may be sealed (or sealable) with a septum cap 1610 that may provide a self-sealing aperture through which a needle or other form of tube of relatively small diameter tube may be inserted to effect the transfer of gases and/or liquids into and/or out of the interior volume of the first cylinder 1601. The other end of the first cylinder 1601 may incorporate a membrane filter 1650. In some embodiments, the membrane filter 1650 may have a molecular weight cutoff ranging from 100 kD to 500 kD.

The second cylinder 1602 may be configured to make the isolator 1600 more amenable for use with the centrifuge 1500. More specifically, one end of the second cylinder 1602 may be closed off with a conical end to ease insertion into the centrifuge 1500, while the other end may be open to enable the two cylinders 1601 and 1602 to be assembled by inserting part of the end of the first cylinder 1601 that includes the membrane filter 1650 therein.

It should be noted (and as depicted) that, in some embodiments, the isolator 1600 may be of an extended length variant 1600a in which the volume of the first cylinder 1601 is increased by sealing the end opposite the membrane filter 1650 with an extended variant of the septum cap 1610 that provides a cylindrical extension 1611 to the cylindrical wall of the first cylinder 1601 to increase the length of the first cylinder 1601. Alternatively, in other embodiments, the isolator 1600 may be of a standard length variant 1600b in which the volume of the first cylinder 1601 is not so increased. More precisely, instead of sealing the end opposite the membrane filter 1650 with the extended variant of the septum cap 1610, a standard variant of the septum cap 1610 is used in the standard length variant 1600b that does not provide the cylindrical extension 1611 of the extended length variant 1600a.

As additionally depicted, the isolator 1600 may carry an ID tag 5600 that is indicative of identity of the patient 90 (or donor 10). In a manner similar to the ID tags 5200, it may be that the ID tag 5600, regardless of the technology it is based upon, may be printed and/or caused to store an identifier at a time on or about the time that the whole blood 11 is drawn from the patient 90 (or donor 10). In this way, like the ID tags 5200, the ID tag 5600 is caused to store an indication of the identity of the individual from which the whole blood 11 is drawn.

Turning to FIG. 2I, with the isolator 1600a or 1600b assembled, and with the transfer needle 1411 connected to end connector 1410 of the transfer syringe 1400, the transfer needle 1411 may then be inserted through the aperture of the septum cap 1610. The plunger 1440 of the transfer syringe 1400 may then be operated to transfer the plasma 13 out of the transfer syringe 1400, and into the first cylinder 1601 through the transfer needle 1411 and the aperture of the septum cap 1610.

Turning to FIG. 2J, after the plasma 13 has been transferred into the first cylinder 1601, the still assembled isolator 1600 may then be placed within the centrifuge 1500 to be subjected to centrifugal force for a second period of time that is deemed sufficient to fully separate the α2M molecules 17 from the rest of the plasma 13 originally transferred into the first cylinder 1601. More specifically, and as depicted, the centrifuge 500 may be used in conjunction with the membrane filter 1650 to effect such a separation of components of the plasma 13. Thus, when such isolation of the α2M molecules 17 is complete, the α2M molecules 17 should remain within the first cylinder 1601, while the other components of the plasma 13 should be retained within the second cylinder 1602 as the depicted waste plasma 14.

In a manner similar to what was discussed in reference to FIG. 2B, the centrifuge 1500 may include a rotor 1556 that defines a pair of holding positions 1560 that each have a shape and dimensions selected to hold a tube of matching shape and dimensions, such as the isolator 1600. As also depicted, it may be that the pair of such holding positions 1560, as defined by the rotor 556, may be positioned to enable the placement of a pair of the isolators 1600 at locations that distribute the weight thereof in a balanced manner that enables relatively smooth operation of the centrifuge 1500.

It should be noted that, although a variant of the rotor 1556 that provides a pair of the holding positions 1560 is depicted and described herein, other embodiments are possible in which the rotor 1556 may have more or fewer of such holding positions 1560. Further, to address situations in which the centrifuge is to be operated with a variant of the rotor 1556 that includes a quantity of holding positions 1560 that differ from the quantity of isolators 1600 that are to be inserted therein, one or more dummy weights of a shape, size and/or weight similar to an isolator 1600 may be used to enable balancing of the centrifuge 1500. Alternatively, an extra isolator 1600 filled with water may be used to serve such a purpose.

As will also be familiar to those skilled in the art, it may be that the depicted rotor 1556 is exchangeable with one or more other rotors (e.g., the rotor 1552 of FIG. 2B) to thereby enable the centrifuge 1500 to be reconfigured to work with various different quantities and/or combinations of various tubes and/or other varieties of containers of differing shapes and/or sizes. Alternatively or additionally (and as was earlier discussed in reference to FIG. 2B), it may be that the centrifuge 1500 is fitted with (or otherwise includes) a rotor with 2 or more “buckets.” Each such bucket may be able to be fitted with any of a variety of differing types of holder that may each be designed to provide holding position(s) for a differing quantity of and/or combination of various tubes and/or other varieties of containers of differing shapes and/or sizes.

It should be noted that such use of the isolator 1600 with the centrifuge 1500 to perform the separation of the α2M molecules 17 from the rest of the plasma 13 originally transferred into the first cylinder 1601 has been found to provide a simpler approach than a peristaltic pump. The centrifuge 1500 is also a far simpler and far less expensive piece of equipment than a peristaltic pump. As a result, the enrichment system 1000 may be more suitable for being carried by and/or installed within a vehicle used to make house calls.

Turning to FIG. 2K, following such isolation of the α2M molecules 17, the α2M molecules 17 may be transferred to the set of vials 800 via the one or more α2M syringes 1700, thereby defining the set of aliquots of the α2M molecules 17 derived from the whole blood 11 drawn in FIG. 2A. More specifically, a needle 1711 connected to an end connector 1710 of each of the one or more α2M syringes 1700 may be inserted through the aperture of the septum cap 1610. With the needle 1711 of each of the one or more α2M syringes 1700 so inserted, a plunger 1770 thereof may then be operated to retrieve the α2M molecules 17 from within the first cylinder 1601, and to subsequently transfer the α2M molecules 17 to the set of vials 800. Where more than one isolator 1600 is used to isolate the α2M molecules 17, such operations may be repeated to transfer the α2M molecules 17 out of each such isolator 1600.

It is envisioned that the amount of the α2M molecules 17 that are isolated from the whole blood 11 will be more than enough to fill more than one of the vials 800. In some embodiments, it may be deemed desirable to provide an amount within each of the vials 800 that is large enough to support multiple administrations of a dose, or of multiple doses, to the patient 90 (e.g., multiple administrations over the course of one or more hours). However, in other embodiments, it may be deemed desirable to provide an amount within each of the vials 800 that is small enough to support just a single dose or single administration of a dose to the patient 90, thereby enabling each dose or administration of a dose to be kept frozen in a separate vial 800 until the time comes that it is needed. Thus, it is envisioned that each aliquot is to be sized to enable one of the aliquots to be used to deliver a first amount to the patient 90, while one or more other aliquots may remain stored in a freezer to be preserved for the later instances of delivery to the same patient 90. In this way, the provision of multiple administrations and/or doses over the course of multiple days, weeks, months, etc. may be more easily supported.

As additionally depicted, each of the vials 800 may carry an ID tag 5800 that is indicative of identity of the individual from which the whole blood 11 was originally drawn. In a manner similar to the ID tags 5200 and 5600, it may be that the ID tags 5800, regardless of the technology on which they are based, may be printed and/or caused to store an identifier at a time on or about the time that the whole blood 11 is drawn from the patient 90 (or donor 10).

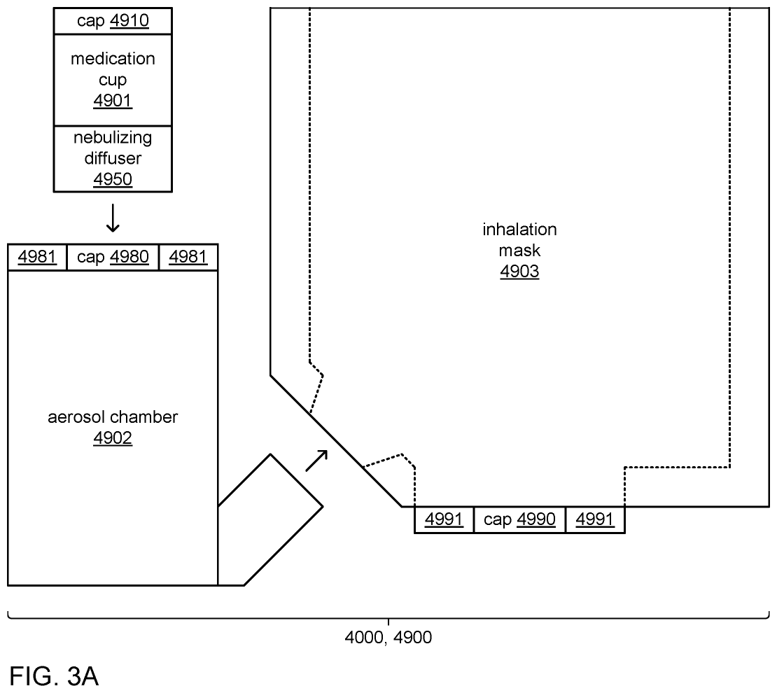

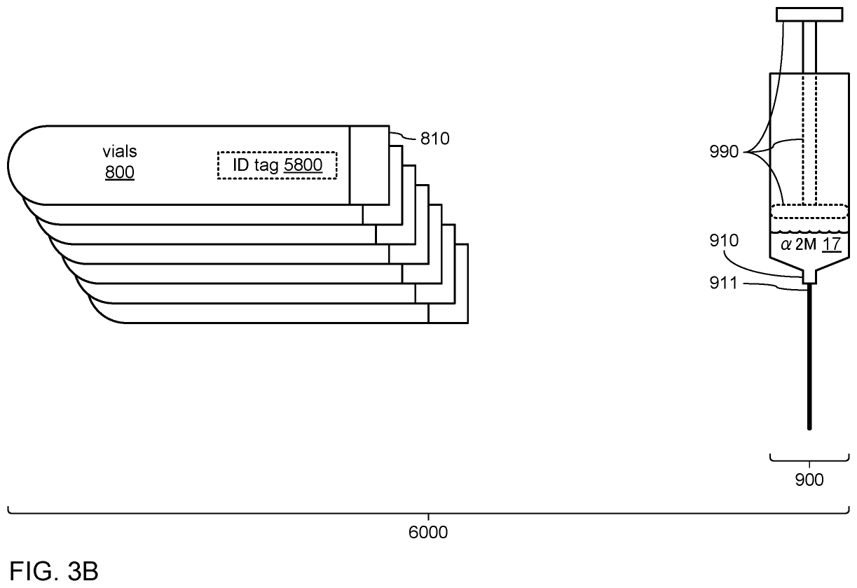

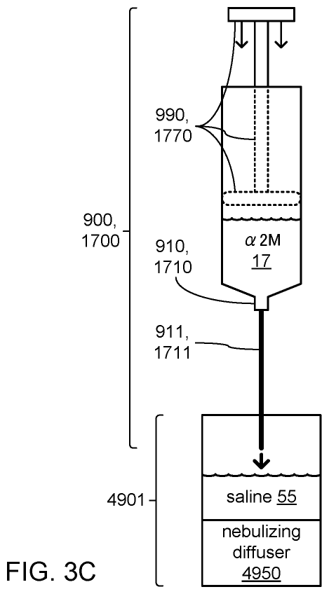

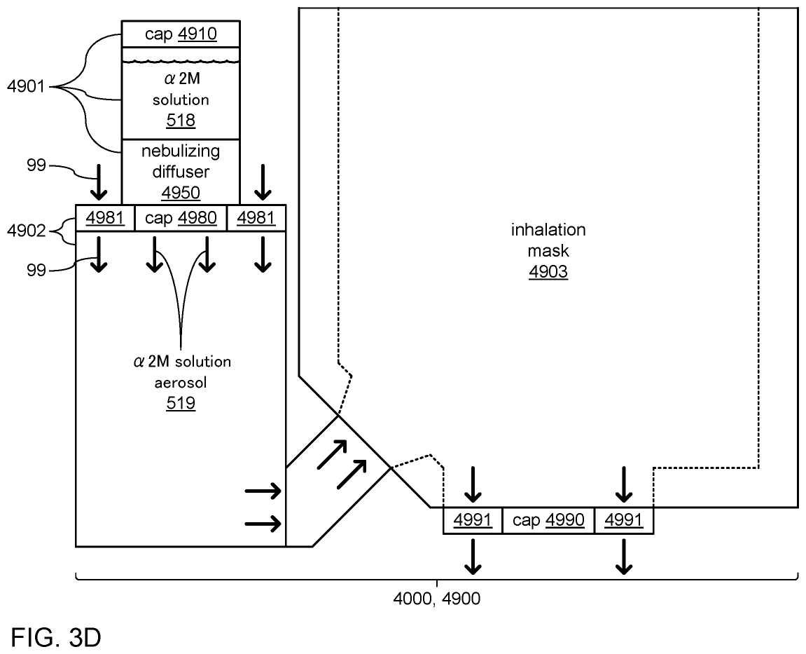

FIGS. 3A-D, taken together, present aspects of the inhalation system 4000 in greater detail. More specifically, FIG. 3A introduces an embodiment of an nebulizer 4900 of the inhalation system 4000; FIGS. 3B-C A-B depict aspects of the provision of the α2M molecules 17 to the nebulizer 4900; and FIG. 3D depicts aspect of the use of the nebulizer 4900 to administer α2M molecules 17 to the patient 90.

Turning to FIG. 3A, as depicted, the nebulizer 4900 of the inhalation system 4000 may include a combination of a medication cup 4901, an aerosol chamber 4902 and an inhalation mask 4903. Both of the medication cup 4901 and the aerosol chamber 4902 may be of a generally elongate cylindrical shape that defines a pair of ends. It should be noted that particular example of the nebulizer 4900 that is depicted may be physically shaped and/or sized for use with such creatures as a camel or horse. More specifically, the inhalation mask 4903 may be physically configured to fit over the muzzle or snout of the patient 90 where the patient 90 is such a creature to thereby intercept the flow of inhalation of air as part of adding a flow of aerosol thereto. Still more specifically, in some embodiments, the nebulizer 4900 may be one of various models offered by FLEXINEB® of Union City, Tennessee, USA. However, other embodiments are possible in which the nebulizer 4900 may be physically shaped and/or sized for use with other creatures having mouths, muzzles, noses, snouts, etc. that may differ considerably from those of a camel or horse.

One end of the medication cup 4901 may be closeable with a cap 4910. The other end of the medication cup 4901 may incorporate a nebulizing diffuser 4950. In various embodiments, the nebulizing diffuser 4950 may employ any of a variety of techniques to generate an aerosol of whatever liquid may be placed within the interior volume of the medication cup 4901 between the cap 4910 and the nebulizing diffuser 4950. Such techniques for aerosolizing such liquids, include, but are not limited to, heating, ultrasonic vibration, etc.

One end of the aerosol chamber 4902 may be closeable with a cap 4980 that may be configured to enable the connection of the end (or other portion) of the medication cup 4901 that incorporates the nebulizing diffuser 4950 thereto in a manner that allows aerosols generated by the nebulizing diffuser 4950 to enter the interior volume of the aerosol chamber 4902. The cap 4980 may additionally include one or more inhalation inlets 4981 that may incorporate one-way valves that enable the entry of surrounding air into the aerosol chamber 4902, while preventing (or at least restricting) the release of the aerosols generated by the nebulizing diffuser 4950 into the surrounding air. The other end of the aerosol chamber 4902 may be configured to be connected to the inhalation mask 4903 in a manner that allows relatively free passage of air and aerosols from within the aerosol chamber 4902 and into the inhalation mask 4903.

The inhalation mask 4903 may incorporate an opening configured to allow the muzzle or snout of such a creature as a camel or a horse (i.e., where the patient 90 is such a creature) to enter into the interior space thereof, and may be further configured for connection to the aerosol chamber 4902 to receive air and aerosols therefrom. There may be still another opening formed in the inhalation mask 4903 that may be closeable with a cap 4990 that may additionally include one or more exhalation outlets 4991 that may incorporate one-way valves that enable the release of gases exhaled from the nose and/or mouth of the muzzle or snout of the patient 90 into the surrounding air, while preventing (or at least restricting) the entry of the surrounding air directly into the inhalation mask 4903. In this way, when then patient 90 inhales while wearing the inhalation mask 4903, the inhaled air is forced to pass through the aerosol chamber 4902, thereby enabling an aerosol generated by the nebulizing diffuser 4950 to be drawn into the inhalation mask 4903 along with the inhaled air.

Turning to FIG. 3B, following the isolation of some of the α2M molecules 17 from the whole blood 11, as described in reference to FIGS. 2A-K, a portion of such α2M molecules 17 may be prepared for being transferred to the nebulizer 4900.

More specifically, and referring back to FIG. 2K in addition to FIG. 3B, it may be that a portion of the α2M molecules 17 that are isolated from the whole blood 11 are to be administered to the patient 90 by inhalation immediately during the same visit in which such isolation is performed. In such a situation, it may be that the very same α2M syringe 1700 (or one of the very same α2M syringes 1700) that is used to transfer α2M molecules 17 into the set of vials 800 is used to transfer some of those α2M molecules 17 to the nebulizer 4900.

Alternatively, and as depicted in FIG. 3B, it may be that at least some of the α2M molecules 17 that are stored within at least one of the vials 800 are to be administered to the patient 90 by inhalation. In such a situation, it may be that a different α2M syringe 900 is used to transfer α2M molecules 17 from one or more of the set of vials 800, and into the nebulizer 4900.

It is envisioned that the amount of the α2M molecules 17 that are isolated from the whole blood 11 will be more than enough to support multiple instances of administering α2M molecules 17 to the patient 90 via inhalation. Thus, it is envisioned that at least one vial 800 will be filled with an aliquot of the α2M molecules 17 that is not meant to be used immediately in an administration of α2M molecules 17 to the patient 90.

Experiments conducted so far by Applicant in treating horses for EA have shown considerable effectiveness with 6 autologous doses delivered at a rate of 1 dose every other day. As will shortly be explained, in these experiments, each dose is diluted with an equal volume of either 0.9% saline solution or lactated Ringer's solution. In these studies, all other medications and/or treatments for EA (if any) were discontinued for all horses. The results were promising, with most of the horses showing improvement. One horse experienced a transient episode of increased coughing associated with one instance of nebulization, and no horses demonstrated epistaxis. Subsequently, many of the horses demonstrated a decrease in clinical signs of EA for a period of 1 to 6 months, and while not being provided with any other medications. Several of the horses that began the study with more severe cases of EA have been able to be maintained with a single nebulization once every 4 weeks. A small number of these horses have additionally been provided with intermittent doses of medications to treat specific clinical signs associated with EA.

It should be noted that further experiments are planned by Applicant to attempt to derive more optimal parameters for the delivery of treatment, including and not limited to, the quantity of α2M molecules 17 to be delivered overall and/or for each aliquot, the number of doses (aliquots) to be delivered and/or their frequency, the ratio of mixture with saline solution and/or another form of dilution liquid, etc.

Turning to FIG. 3C, as part of preparing to use the nebulizer 4900 to deliver a dose of the isolated α2M molecules 17, the cap 4910 may be removed from the medication cup 4901 to provide access to the interior volume therein, and an amount of saline solution 55 may be deposited therein. Also, an amount of the isolated α2M molecules 17 (e.g., some or all of an aliquot contained within one of the vials 800) may also be deposited within the interior volume of the medication cup 4901. As previously discussed, successful results in treating at least EA have been achieved through use of an equal parts mixture of isolated α2M molecules 17 and 0.9% saline solution. However, as also previously discussed, further planned experimentation may demonstrate that a different mixture ratio and/or the use of a different solution begets still better results.

Turning to FIG. 3D, after the isolated α2M molecules 17 and the saline solution 55 have been deposited within the medication cup 4901, thereby creating an α2M solution 518, the cap 4910 may be reinstalled thereon, and the medication cup 4901 may be coupled to the cap 4980 of the aerosol chamber 4902. With the aerosol chamber 4902 also connected to the inhalation mask 4903, and with the inhalation mask 4903 being worn by the patient 90, the nebulizing diffuser 4950 may then be operated to begin generating an α2M solution aerosol 519 from the α2M solution 518. As the α2M solution aerosol 519 is so generated, it is released into the aerosol chamber 4902, thereby building up an amount thereof that is retained within the aerosol chamber 4902 until the patient 90 takes a breath. This results in much of the α2M solution aerosol 519 being drawn, along with surrounding air 99 that enters into the aerosol chamber 4902 through inhalation inlets 4981, into the inhalation mask 4903, where the patient 90 inhales both.

As the patient 90 exhales, the valves within the inhalation inlets 4981 and/or within the exhalation outlets 4991 cooperate with the pressure behind the exhalation by the patient 90 to stop the flow of air 99 and α2M solution aerosol 519 into the inhalation mask 4903 from the aerosol chamber 4902, thereby allowing another amount of the α2M solution aerosol 519 to collect within the aerosol chamber 4902 in preparation for the next inhalation by the patient 90. The valves within the inhalation inlets 4981 and/or the exhalation outlets 4991 also cooperate with the pressure behind the exhalation by the patient 90 to cause the exhaled gases from the patient 90 to be released from within the inhalation mask 4903 through the exhalation outlets 4991.

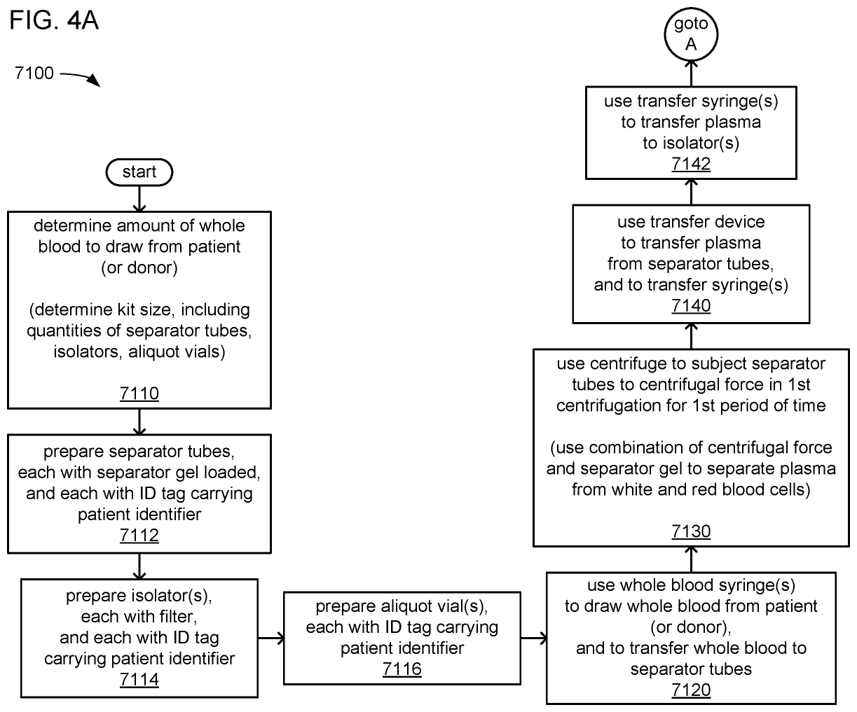

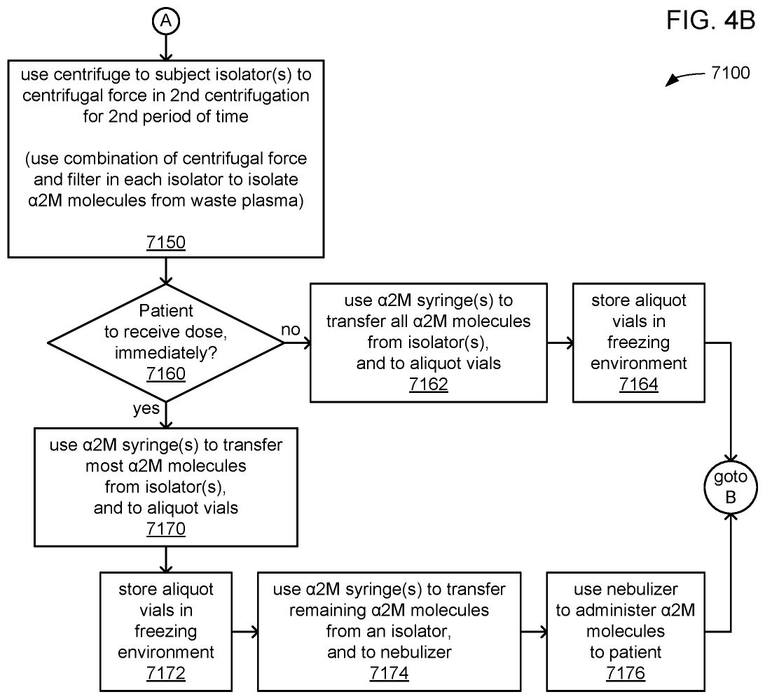

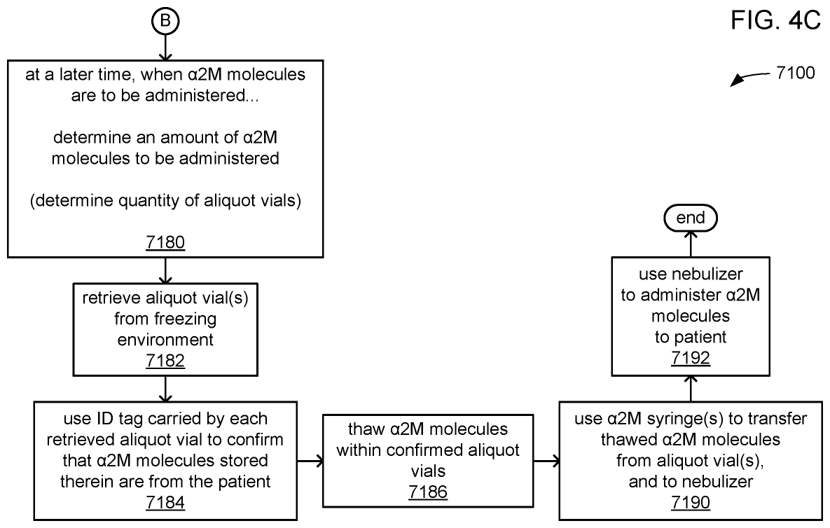

FIGS. 4A-C, taken together, present a flowchart 7100 depicting aspects of the operation of an example of the system of FIG. 1B (and also of FIGS. 2A-K and 3A-D) for isolating, concentrating and administering α2M molecules in an autologous manner via inhalation.

At 7110, the amount of whole blood required to treat a respiratory condition of a patient (e.g., the patient 90—e.g., a camel, a dog, a horse, a human, a pig, etc.) may be determined. More precisely, a kit may be selected that includes sufficient quantities of separator tubes, isolators and aliquot vials to support the provision of enough α2M molecules to treat the patient from the whole blood of the patient (e.g., the α2M molecules 17 from the whole blood 11 of the patient 90, or of a donor 10).

At 7112, the separator tubes may be prepared with separator gel deposited within each, and with each carrying an ID tag that carries an identifier of the patient (e.g., the separator tubes 1200, each with separator gel 1250 therein, and each carrying an ID tag 5200). At 7114, each isolator of the one or more isolators may be prepared with a filter carried within each, and with each carrying an ID tag that carries the identifier of the patient (e.g., the one or more isolators 1600, each with a filter 1650 therein, and each carrying an ID tag 5600). At 7116, the aliquot vials may be prepared with each carrying an ID tag that carries the identifier of the patient (e.g., the aliquot vials 800, each carrying an ID tag 5800). Alternatively, and as previously discussed, each such ID tag carried by a separator tube, an isolator or an aliquot vial, may carry the identifier of a donor in an embodiment where the whole blood is drawn from a donor, instead of from the patient.

At 7120, one or more whole blood syringes may be used to draw whole blood from the patient (e.g., the one or more whole blood syringes 100), and to transfer that whole blood to the separator tubes. Again, as an alternative, the blood may be drawn from a donor using such syringes, instead of from the patient.

At 7130, the separator tubes may be placed within a centrifuge (e.g., the centrifuge 1500), and the centrifuge may be operated to exert centrifugal force on the separator tubes in a first stage of centrifuging (i.e., a first centrifugation) for a first period of time. In this way, a combination of the exerted centrifugal force and the separator gel within each separator tube may be used to separate the plasma containing α2M molecules from other components of the whole blood (e.g., separating the plasma 13 from the red and white blood cells 12).

At 7140, a transfer device may be used to transfer the plasma from within each of the separator tubes to one or more transfer syringes (e.g., one of the transfer devices 1300a, 1300b or 1300c, to one or more of the transfer syringes 1400). At 7142, the one or more transfer syringes may be used to transfer the plasma into one or more isolators.

At 7150, the one or more isolators may be placed within the centrifuge, and the centrifuge may be operated to exert centrifugal force on the isolator(s) in a second stage of centrifuging (i.e., a second centrifugation) for a second period of time. In this way, a combination of the exerted centrifugal force and the filter within each isolator that is filled with plasma may be used to isolate the α2M molecules from other components of the plasma. Again, where just one isolator is filled with plasma, a counterbalancing weight, or other isolator that is filled with water or another substance to serve as a counterbalancing weight, may be required to balance the centrifuge.

If, at 7160, the patient is not to receive a dose of the α2M molecules, immediately, then at 7162, one or more α2M syringes may be used to transfer all of the α2M molecules from the isolator(s), and to the aliquot vials (e.g., the α2M syringes 1700 transferring α2M molecules 17 to the vials 800). With the α2M molecules so transferred to the aliquot vials, at 7164, the aliquot vials may be stored within a freezing environment (e.g., a freezer) to preserve the α2M molecules in storage for an extended period of time, or until needed to treat the patient.

However, if, at 7160, the patient is to receive a dose of the α2M molecules, immediately, then at 7170, one or more α2M syringes may be used to transfer most of the α2M molecules from the isolator(s), and to the aliquot vials, followed by storing the aliquot vials within a freezing environment at 7172. At 7174, a single α2M syringe with an amount of the α2M molecules appropriate for a dose remaining therein may be used to transfer that remaining amount of the α2M molecules to a nebulizer (e.g., the nebulizer 4900 of the inhalation system 4000). With that amount of α2M molecules so transferred to the nebulizer, at 7176, the nebulizer may be used to administer that amount of the α2M molecules to the patient via inhalation.

Regardless of whether the patient is immediately provided with a dose at 7176, or the α2M molecules are simply stored in vials in a freezing environment at 7164 without providing an immediate dose to the patient, it may be that a dose is to be administered to the patient at a later time. Thus, at 7180, at such a later time, an amount α2M molecules to administer to the patient in that dose may be determined.

At 7182, a quantity of aliquot vials required to provide the dose to the patient may be retrieved from the freezing environment in which the aliquot vials were stored. At 7184, the ID tag carried by each retrieved aliquot vial may be used to confirm that the α2M molecules stored therein are from the patient. At 7186, each of such retrieved and confirmed aliquot vials may be thawed as part of preparing the α2M molecules stored therein for being administered to the patient.

At 7190, an α2M syringe may be used to transfer the α2M molecules stored within each of the thawed aliquot vials to the nebulizer. With those α2M molecules so transferred to the nebulizer, at 7192, the nebulizer may be used to administer those α2M molecules to the patient via inhalation.

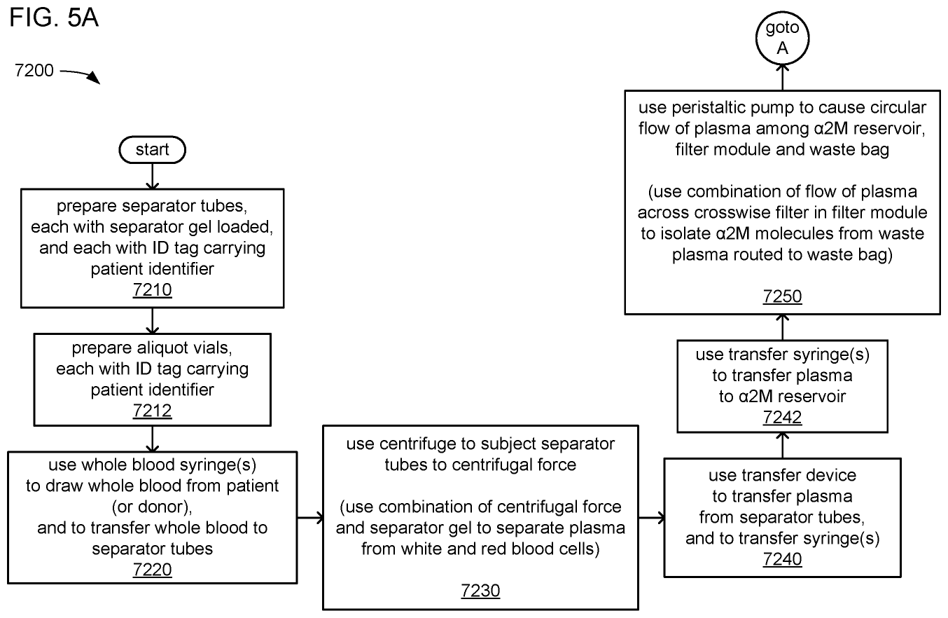

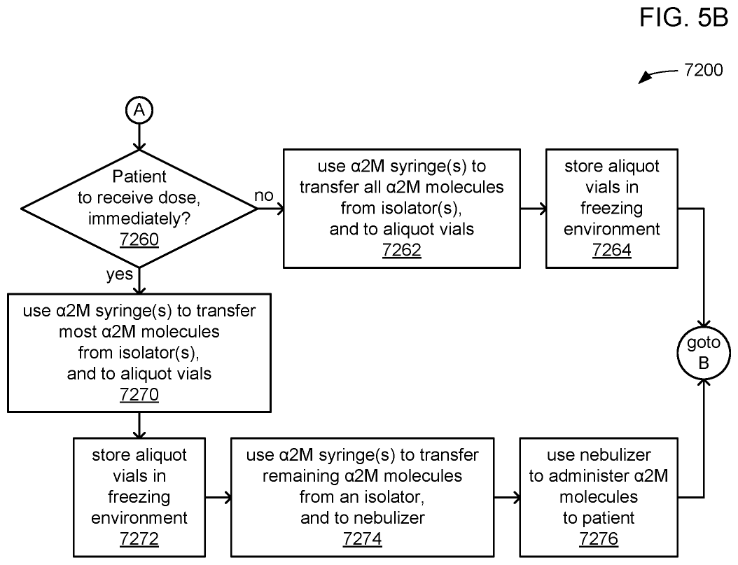

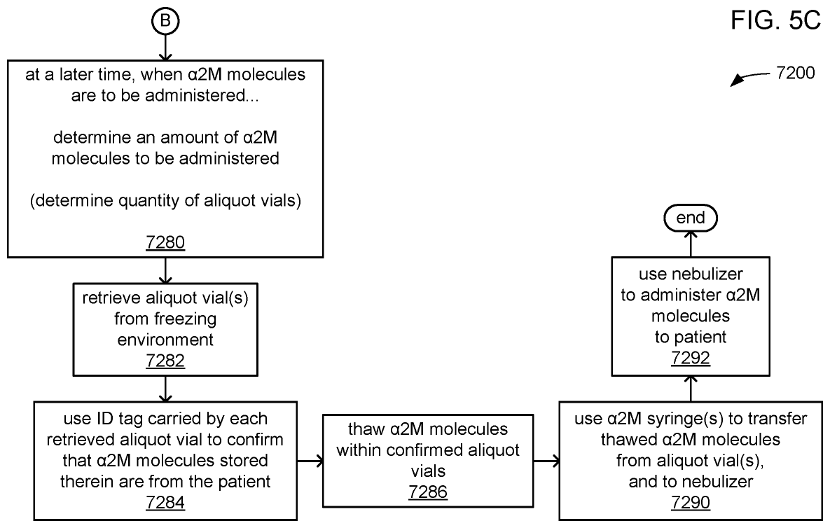

FIGS. 5A-C, taken together, present a flowchart 7200 depicting aspects of the operation of an example of the system of FIG. 1D for isolating, concentrating and administering α2M molecules in an autologous manner via inhalation.

At 7210, a quantity of separator tubes may be prepared with separator gel deposited within each, and with each carrying an ID tag that carries an identifier of the patient (e.g., the separator tubes 1200, each with separator gel 1250 therein, and each carrying an ID tag 5200). At 7212, a quantity of aliquot vials may be prepared with each carrying an ID tag that carries the identifier of the patient (e.g., the aliquot vials 800, each carrying an ID tag 5800). Alternatively, and as previously discussed, each such ID tag carried by a separator tube or an aliquot vial, may carry the identifier of a donor in an embodiment where the whole blood is drawn from a donor, instead of from the patient.

At 7220, one or more whole blood syringes may be used to draw whole blood from a patient (e.g., the one or more whole blood syringes 100 used to draw whole blood 11 of the patient 90), and to transfer that whole blood to the separator tubes. Again, as an alternative, the blood may be drawn from a donor using such syringes, instead of from the patient.

At 7230, the separator tubes may be placed within a centrifuge (e.g., the centrifuge 1500), and the centrifuge may be operated to exert centrifugal force on the separator tubes. In this way, a combination of the exerted centrifugal force and the separator gel within each separator tube may be used to separate the plasma containing α2M molecules from other components of the whole blood (e.g., separating the plasma 13 from the red and white blood cells 12).

At 7240, a transfer device may be used to transfer the plasma from within each of the separator tubes to one or more transfer syringes (e.g., one of the transfer devices 1300a, 1300b or 1300c, to one or more of the transfer syringes 1400). At 7242, the one or more transfer syringes may be used to transfer the plasma into an α2M reservoir (e.g., the α2M reservoir 2600).

At 7250, the α2M reservoir may be connected to a filtration module and a waste bag (e.g., the filtration module 2300 and the waste bag 2400), and a peristaltic pump may then be used to circulate the plasma among the α2M reservoir, a crosswise filter of the filter module and the waste bag. In this way, the plasma is repeated circulated through the filter module for a period of time sufficient to cause α2M molecules to remain within the α2M reservoir, while other components of the plasma pass through the crosswise filter of the filter module, and into the waste bag.

At 7260, and proceeding to 7292, the rest of the flowchart 7200 may proceed in a manner similar to what has been described above from 7160 to 7192 of the flowchart 7100.

There is thus disclosed a system and method for the isolation, concentration and delivery of Alpha-2 Macroglobulin (α2M) molecules to treat respiratory conditions.

A method for treating a respiratory condition of a patient with Alpha-2 Macroglobulin (α2M) molecules includes: drawing whole blood; separating plasma containing α2M molecules from other components of the whole blood; isolating the α2M molecules from the other components of the plasma; and administering at least some of the isolated α2M molecules to the patient via inhalation.

The whole blood may be drawn from the patient in support of treating the patient with the α2M molecules in an autologous manner.

Drawing the whole blood may include: using a whole blood syringe comprising a hollow needle to draw the whole blood; and partially pre-filling the whole blood syringe with an anticoagulant before using the whole blood syringe to draw the whole blood.

The anticoagulant may include a citrate dextrose solution (ACD-A).

Separating the plasma from other components of the whole blood may include: depositing the whole blood into at least one separator tube, wherein each separator tube of the at least one separator tube contains an amount of separator gel; and subjecting the at least one separator tube to a first centrifugal force in a first centrifuging stage for a first predetermined period of time to cause a combination of the first centrifugal force and the separator gel within each separator tube of the at least one separator tube to separate the plasma of the whole blood within the at least one separator tube from red blood cells and white blood cells of the whole blood within the at least one separator tube.

Each separator tube of the at least one separator tube may include a vacuum separator tube that is pre-provided with a vacuum therein when in an unused condition.

Subjecting the at least one separator tube to the first centrifugal force in the first centrifuging stage may include placing the at least one separator tube within a first holder of a centrifuge, and the first holder may include either a first removable holder configured to be inserted into a bucket of the centrifuge, or a first exchangeable rotor of the centrifuge

Isolating the α2M molecules from the other components of the plasma may include: following the first centrifuging stage, transferring the plasma from the at least one separator tube and into at least one isolator, wherein each isolator of the at least one isolator comprises a filter; and subjecting the at least one isolator to a second centrifugal force in a second centrifuging stage for a second predetermined period of time to cause a combination of the second centrifugal force and the filter within each isolator of the at least one isolator to isolate the α2M molecules from the other components of the plasma within the at least one isolator.

Transferring the plasma from the at least one separator tube and into the at least one isolator may include: coupling a transfer syringe to a syringe port of a transfer device, wherein the syringe port is configured to receive an end connector of the transfer syringe that is configured to be coupled to a transfer needle; coupling each separator tube of the at least one separator tube, one at a time, to a separator tube port of the transfer device, wherein the separator tube port comprises at least one hollow needle configured to penetrate the cap of each separator tube to couple the separator tube to the syringe port of the transfer device; while each separator tube of the at least one separator tube is coupled to the separator tube port, operating a plunger of the transfer syringe to withdraw at least some of the plasma from within the separator tube and into the transfer syringe through the transfer device; and following transfer of plasma from each separator tube of the at least one separator tube, using the transfer syringe, with the transfer needle coupled to the end connector, to inject the plasma within the transfer syringe into the at least one isolator.

The filter of each isolator of the at least one isolator may have a molecular weight cut off ranging from 100 kD to 500 kD.

Isolating the α2M molecules from the other components of the plasma may include: following the first centrifuging stage, transferring the plasma from the at least one separator tube and into an α2M reservoir; and using a peristaltic pump to circulate the plasma among the α2M reservoir, a crosswise filter and a waste bag to cause other components of the plasma to pass through the crosswise filter and into the waste bag, while the α2M molecules remain within the α2M reservoir.

Administering at least some of the isolated α2M molecules to the patient via inhalation may include administering at least some of the isolated α2M molecules to the patient using a nebulizer.

The method may further include storing a remainder of the isolated α2M molecules within at least one vial in a freezing environment to preserve the remainder of the isolated α2M molecules for use in another administration of the isolated α2M molecules to the patient at a later time.

A kit for treating a respiratory condition of a patient with Alpha-2 Macroglobulin (α2M) molecules includes at least one separator tube, wherein each separator tube of the at least one separator tube includes: an elongate transparent tube that defines an opening at one end that is sealed with a cap that is penetrable to receive whole blood; and an amount of separator gel disposed within the separator tube to cooperate with a first centrifugal force exerted on the separator tube for a first period of time during a first centrifuging stage to separate plasma containing α2M molecules from other components of the whole blood. The kit also includes at least one isolator, wherein each isolator of the at least one isolator includes: a filter; a first cylinder defined by a first cylindrical wall having a first end that is configured to be closable with a septum cap that is penetrable to receive the plasma containing the α2M molecules following the first centrifuging stage, and having a second end that is closed with the filter; and a second cylinder defined by a second cylindrical wall having a first end that is closed where the second cylindrical wall narrows to form a conically-shaped end portion, and having a second end that defines an opening that is configured to be coupled to the filter in a manner that causes a first interior space of the first cylinder and a second interior space of the second cylinder to be separated by the filter, wherein the filter is configured to cooperate with a second centrifugal force exerted on the isolator for a second period of time during a second centrifuging stage to isolate the α2M molecules from other components of the plasma. The further includes a transfer device that includes: a separator tube port configured to receive each separator tube of the at least one separator tube, one at a time, wherein the separator tube port comprises at least one hollow needle configured to penetrate the cap of each separator tube to couple the separator tube to a syringe port of the transfer device; and a syringe port configured to receive an end connector of a transfer syringe that is configured to be coupled to a transfer needle, wherein, following the first centrifuging stage and prior to the second centrifuging stage, while each separator tube of the at least one separator tube is coupled to the separator tube port, a plunger of the transfer syringe is operable to withdraw at least some of the plasma from within the separator tube and into the transfer syringe through the transfer device, and following transfer of plasma from each separator tube of the at least one separator tube, and with the transfer needle coupled to the end connector to penetrate the septum cap of each isolator of the at least one isolator, the plunger of the transfer syringe is operable to inject the plasma within the transfer syringe into the at least one isolator. The kit still further includes a nebulizer configured to be provided with the α2M molecules isolated during the second centrifuging stage, and to administer the α2M molecules to the patient via inhalation.

The whole blood may be drawn from the patient in support of treating the patient with the α2M molecules in an autologous manner.

The kit may further include a whole blood syringe configured to draw the whole blood, wherein the whole blood syringe may include an amount of an anticoagulant carried within the whole blood syringe to prevent the whole blood from coagulating therein.

The anticoagulant may include a citrate dextrose solution (ACD-A).

Each separator tube of the at least one separator tube may include either: a non-vacuum separator tube; or a vacuum separator tube that, when in an unused condition, is pre-provided with a vacuum therein that the seal provided by the cap is used to maintain.

The kit may further include a centrifuge, wherein the centrifuge may include at least one exchangeable rotor to enable the centrifuge to be used in the first centrifuging stage and the second centrifuging stage by exchanging the at least one exchangeable rotor.

The kit may further include: a centrifuge including a rotor that defines multiple buckets; and a first set of exchangeable holders and a second set of exchangeable holders, wherein the multiple buckets enable the centrifuge to be used in the first centrifuging stage with the first set of exchangeable holders installed within the multiple buckets, and enable the centrifuge to be used in the second centrifuging stage with the second set of exchangeable holders installed within the multiple buckets.

Each isolator of the at least one isolator may be configured to: receive the injection of the plasma within the first interior space within the first cylinder; and isolate the α2M molecules within the first interior space from the other components of the plasma within the second interior space.

The septum cap may further include a third cylindrical wall configured to serve as an extension to the first cylindrical wall to increase a volume of the first interior space when the first end of the first cylindrical wall is closed with the septum cap.

The filter of each isolator of the at least one isolator may have a molecular weight cut off ranging from 100 kD to 500 kD.

The at least one isolator may include just one isolator; and the kit may further include a centrifuge and a dummy isolator configured to cooperate with the centrifuge to provide a counterbalance to the just one isolator when subjecting the just one isolator during the second centrifuging stage.

1. A method for treating a respiratory condition of a patient with Alpha-2 Macroglobulin (α2M) molecules, the method comprising:
drawing whole blood;
separating plasma containing α2M molecules from other components of the whole blood;
isolating the α2M molecules from the other components of the plasma; and
administering at least some of the isolated α2M molecules to the patient via inhalation.

2. The method of claim 1, wherein the whole blood is drawn from the patient in support of treating the patient with the α2M molecules in an autologous manner.

3. The method of claim 1, wherein drawing the whole blood comprises:
using a whole blood syringe comprising a hollow needle to draw the whole blood; and
partially pre-filling the whole blood syringe with an anticoagulant before using the whole blood syringe to draw the whole blood.

4. The method of claim 3, wherein the anticoagulant comprises a citrate dextrose solution (ACD-A).

5. The method of claim 1, wherein separating the plasma from other components of the whole blood comprises:
depositing the whole blood into at least one separator tube, wherein each separator tube of the at least one separator tube contains an amount of separator gel; and
subjecting the at least one separator tube to a first centrifugal force in a first centrifuging stage for a first predetermined period of time to cause a combination of the first centrifugal force and the separator gel within each separator tube of the at least one separator tube to separate the plasma of the whole blood within the at least one separator tube from red blood cells and white blood cells of the whole blood within the at least one separator tube.

6. The method of claim 5, wherein each separator tube of the at least one separator tube comprises a vacuum separator tube that is pre-provided with a vacuum therein when in an unused condition.

7. The method of claim 5, wherein:
subjecting the at least one separator tube to the first centrifugal force in the first centrifuging stage comprises placing the at least one separator tube within a first holder of a centrifuge; and
the first holder comprises either a first removable holder configured to be inserted into a bucket of the centrifuge, or a first exchangeable rotor of the centrifuge

8. The method of claim 5, wherein isolating the α2M molecules from the other components of the plasma comprises:
following the first centrifuging stage, transferring the plasma from the at least one separator tube and into at least one isolator, wherein each isolator of the at least one isolator comprises a filter; and
subjecting the at least one isolator to a second centrifugal force in a second centrifuging stage for a second predetermined period of time to cause a combination of the second centrifugal force and the filter within each isolator of the at least one isolator to isolate the α2M molecules from the other components of the plasma within the at least one isolator.

9. The method of claim 8, wherein transferring the plasma from the at least one separator tube and into the at least one isolator comprises:
coupling a transfer syringe to a syringe port of a transfer device, wherein the syringe port is configured to receive an end connector of the transfer syringe that is configured to be coupled to a transfer needle;
coupling each separator tube of the at least one separator tube, one at a time, to a separator tube port of the transfer device, wherein the separator tube port comprises at least one hollow needle configured to penetrate the cap of each separator tube to couple the separator tube to the syringe port of the transfer device;
while each separator tube of the at least one separator tube is coupled to the separator tube port, operating a plunger of the transfer syringe to withdraw at least some of the plasma from within the separator tube and into the transfer syringe through the transfer device; and
following transfer of plasma from each separator tube of the at least one separator tube, using the transfer syringe, with the transfer needle coupled to the end connector, to inject the plasma within the transfer syringe into the at least one isolator.

10. The method of claim 8, wherein the filter of each isolator of the at least one isolator has a molecular weight cut off ranging from 100 kD to 500 kD.

11. The method of claim 1, wherein isolating the α2M molecules from the other components of the plasma comprises:
following the first centrifuging stage, transferring the plasma from the at least one separator tube and into an α2M reservoir; and
using a peristaltic pump to circulate the plasma among the α2M reservoir, a crosswise filter and a waste bag to cause other components of the plasma to pass through the crosswise filter and into the waste bag, while the α2M molecules remain within the α2M reservoir.

12. The method of claim 1, wherein administering at least some of the isolated α2M molecules to the patient via inhalation comprises administering at least some of the isolated α2M molecules to the patient using a nebulizer.

13. The method of claim 1, further comprising storing a remainder of the isolated α2M molecules within at least one vial in a freezing environment to preserve the remainder of the isolated α2M molecules for use in another administration of the isolated α2M molecules to the patient at a later time.

14. A kit for treating a respiratory condition of a patient with Alpha-2 Macroglobulin (α2M) molecules, the kit comprising:
at least one separator tube, wherein each separator tube of the at least one separator tube comprises:
an elongate transparent tube that defines an opening at one end that is sealed with a cap that is penetrable to receive whole blood; and
an amount of separator gel disposed within the separator tube to cooperate with a first centrifugal force exerted on the separator tube for a first period of time during a first centrifuging stage to separate plasma containing α2M molecules from other components of the whole blood;
at least one isolator, wherein each isolator of the at least one isolator comprises:
a filter;
a first cylinder defined by a first cylindrical wall having a first end that is configured to be closable with a septum cap that is penetrable to receive the plasma containing the α2M molecules following the first centrifuging stage, and having a second end that is closed with the filter; and
a second cylinder defined by a second cylindrical wall having a first end that is closed where the second cylindrical wall narrows to form a conically-shaped end portion, and having a second end that defines an opening that is configured to be coupled to the filter in a manner that causes a first interior space of the first cylinder and a second interior space of the second cylinder to be separated by the filter, wherein:
the filter is configured to cooperate with a second centrifugal force exerted on the isolator for a second period of time during a second centrifuging stage to isolate the α2M molecules from other components of the plasma;
a transfer device, comprising:
a separator tube port configured to receive each separator tube of the at least one separator tube, one at a time, wherein the separator tube port comprises at least one hollow needle configured to penetrate the cap of each separator tube to couple the separator tube to a syringe port of the transfer device; and
a syringe port configured to receive an end connector of a transfer syringe that is configured to be coupled to a transfer needle, wherein, following the first centrifuging stage and prior to the second centrifuging stage:
while each separator tube of the at least one separator tube is coupled to the separator tube port, a plunger of the transfer syringe is operable to withdraw at least some of the plasma from within the separator tube and into the transfer syringe through the transfer device; and
following transfer of plasma from each separator tube of the at least one separator tube, and with the transfer needle coupled to the end connector to penetrate the septum cap of each isolator of the at least one isolator, the plunger of the transfer syringe is operable to inject the plasma within the transfer syringe into the at least one isolator; and
a nebulizer configured to be provided with the α2M molecules isolated during the second centrifuging stage, and to administer the α2M molecules to the patient via inhalation.

15. The kit of claim 14, wherein the whole blood is drawn from the patient in support of treating the patient with the α2M molecules in an autologous manner.

16. The kit of claim 14, further comprising a whole blood syringe configured to draw the whole blood, wherein the whole blood syringe comprises an amount of an anticoagulant carried within the whole blood syringe to prevent the whole blood from coagulating therein.

17. The kit of claim 16, wherein the anticoagulant comprises a citrate dextrose solution (ACD-A).

18. The kit of claim 14, wherein each separator tube of the at least one separator tube comprises either:
a non-vacuum separator tube; or
a vacuum separator tube that, when in an unused condition, is pre-provided with a vacuum therein that the seal provided by the cap is used to maintain.

19. The kit of claim 14, further comprising a centrifuge, wherein the centrifuge comprises at least one exchangeable rotor to enable the centrifuge to be used in the first centrifuging stage and the second centrifuging stage by exchanging the at least one exchangeable rotor.

20. The kit of claim 14, further comprising:
a centrifuge comprising a rotor that defines multiple buckets; and
a first set of exchangeable holders and a second set of exchangeable holders, wherein the multiple buckets enable the centrifuge to be used in the first centrifuging stage with the first set of exchangeable holders installed within the multiple buckets, and enable the centrifuge to be used in the second centrifuging stage with the second set of exchangeable holders installed within the multiple buckets.

21. The kit of claim 14, wherein each isolator of the at least one isolator is configured to:
receive the injection of the plasma within the first interior space within the first cylinder; and
isolate the α2M molecules within the first interior space from the other components of the plasma within the second interior space.

22. The kit of claim 14, wherein the septum cap further comprises a third cylindrical wall configured to serve as an extension to the first cylindrical wall to increase a volume of the first interior space when the first end of the first cylindrical wall is closed with the septum cap.

23. The kit of claim 14, wherein the filter of each isolator of the at least one isolator has a molecular weight cut off ranging from 100 kD to 500 kD.

24. The kit of claim 14, wherein:
the at least one isolator comprises just one isolator; and
the kit further comprises a centrifuge and a dummy isolator configured to cooperate with the centrifuge to provide a counterbalance to the just one isolator when subjecting the just one isolator during the second centrifuging stage.
